(19) United States
(12) Patent Application Publication
(10) Pub. No.: US 2025/0345573 A1
(43) Pub. Date: Nov. 13, 2025

(54) BALLOON CATHETER SYSTEM

(71) Applicant: Medtronic Ireland Manufacturing Unlimited Company, Dublin (IE)

(72) Inventors: Gerry Oliver McCaffrey, Abbeyknockmoy (IE); Armindo G. Fonseca, Santa Rosa, CA (US)

(21) Appl. No.: 18/861,090

(22) PCT Filed: Apr. 26, 2023
(86) PCT No.: PCT/EP2023/060991

§ 371 Date: Oct. 28, 2024

Related U.S. Application Data
(60) Provisional application No. 63/363,959, filed on May 2, 2022.

(51) Int. Cl. A61M 25/10; A61N 7/00; A61N 7/02

(52) U.S. Cl. CPC A61M 25/10185; A61M 25/10181; A61M 25/10187; A61N 7/02; A61M 2205/3344; A61N 2007/003

(57) ABSTRACT

A catheter system (108) includes a balloon (112) and control circuitry (128) configured to control a size of the balloon by at least controlling an inflation pressure of the balloon. For example, the control circuitry can be configured to control the inflation pressure by at least controlling a flow of a fluid through an interior volume of the balloon. In some examples, the control circuitry is configured to control the size of the balloon based on an input provided by a user, e.g., based on user input indicating a pressure set point or a particular balloon size. In addition, in some examples, the control circuitry is configured to control an therapeutic element based on the size of the balloon.

### TECHNICAL FIELD

The present technology is related to catheters.

### BACKGROUND

Catheters have been proposed for use with various medical procedures. For example, a catheter can be configured to deliver neuromodulation therapy to a target tissue site to modify the activity of nerves at or near the target tissue site. The nerves can be, for example, sympathetic nerves. The sympathetic nervous system (SNS) is a primarily involuntary bodily control system typically associated with stress responses. Chronic over-activation of the SNS is a maladaptive response that can drive the progression of many disease states. For example, excessive activation of the renal SNS has been identified experimentally and in humans as a likely contributor to the complex pathophysiology of arrhythmias, hypertension, states of volume overload (e.g., heart failure), and progressive renal disease.

### SUMMARY

The present disclosure describes catheter systems including an elongate body and a balloon configured to be positioned within a blood vessel of a patient to deliver a therapy to the patient. The therapy can include, for example, neuromodulation therapy, such as renal denervation. The catheter system includes a therapeutic element (e.g., an ultrasound element) configured to delivery therapy to a vessel wall or other tissues of the patient. The balloon is configured to be expanded within the blood vessel to, for example, assist in occluding the blood vessel during a medical procedure, assist in maintaining the elongate body within the blood vessel, assist in displacing the elongate body and a wall of the blood vessel, assist in positioning the therapeutic element within the blood vessel, and/or for other reasons. For example, the expanded balloon can be configured to approximately center the therapeutic element within the blood vessel, helps retain the therapeutic element in position relative to the vessel wall, and/or assists in other ways. In addition, in some examples, a fluid that is used to expand (also referred to herein as inflate) the balloon is configured to modify a temperature at or near the target tissue site. For example, the therapeutic element can be configured to heat the fluid, which then heats the target tissue site at the balloon and target tissue site interface.

In some examples, the catheter system is configured to control a size of the balloon by at least controlling an inflation pressure of the balloon. The catheter system is configured such that the inflation pressure is dependent on (e.g., a function of) a flow of a fluid (e.g., saline) through an interior volume of the balloon. The catheter system includes control circuitry configured to control the inflation pressure by at least controlling the flow of the fluid through the interior volume. In some examples, the control circuitry is configured to associate a dimension (e.g., a diameter) of the balloon with a particular inflation pressure value or range of inflation pressure values. The control circuitry may be configured to control the flow of the fluid to substantially maintain a particular inflation pressure (or range of inflation pressures), which enables the control circuitry to control the size of the balloon. In some examples, the control circuitry is configured to control the size of the balloon based on an input provided by a clinician or other user.

A size of the balloon defines a displacement between the therapeutic element and a vessel wall when the balloon is inflated within a blood vessel. Due to this separation between the therapeutic element and the blood vessel wall, for larger vessels, more energy may need to be delivered via the therapeutic element to achieve a desired therapeutic outcome compared to smaller blood vessels. Thus, in some examples, an amount of energy (e.g., ultrasound energy or microwave energy) delivered by the therapeutic element can be selected based on a size of the blood vessel in which the catheter system is positioned. Because a clinician may select a size of the balloon (when expanded) such that the balloon is in apposition with the blood vessel wall, in some examples, control circuitry is configured to control the power of the therapeutic element based on information indicative of the size of the expanded balloon. Such information can be provided by a user or can come from another source.

In examples, a catheter system comprises: an elongate body configured to be positioned within a blood vessel of a patient; a balloon defining an interior volume; a sensor configured to generate a pressure signal indicative of a pressure of a fluid within a flow path, the flow path including the interior volume and at least one lumen defined by the elongate body; a valve configured to define a flow area for the fluid; and control circuitry configured to: determine a pressure setpoint, receive the pressure signal from the sensor, and position the valve to adjust the flow area based on the pressure setpoint and the pressure indicated by the sensor signal.

In examples, a catheter system comprises: an elongate body; a balloon defining an interior volume; a therapeutic element mechanically supported by the elongate body, wherein the therapeutic element is configured to emit energy; and control circuitry configured to: receive an input indicative of a pressure within the interior volume of the balloon, determine a power level based on the indicated pressure, and cause the therapeutic element to emit the energy based on the determined power level.

In examples, a method comprises: receiving, by control circuitry, a sensor input from a pressure sensor indicative of a pressure of a fluid within a flow path for a fluid flowing through an interior volume of a balloon, the flow path being defined by an elongated body of a catheter, wherein the elongated structure is configured to be positioned with an anatomical lumen of a patient; and positioning, using the control circuitry, a valve configured to throttle a flow of the fluid through the flow path based on a difference between a pressure setpoint and the pressure indicated by the sensor input.

In examples, a method comprises: receiving, by control circuitry, an input indicative of a pressure within an interior volume of a balloon of a catheter, wherein an elongated body of the catheter defines a flow path for a fluid flowing through the interior volume of the balloon, and wherein at least a distal portion of the catheter is configured to be positioned with an anatomical lumen of a patient; determining, by the control circuitry, a power level of an energy emitting element of the catheter based on the indicative input; and causing, using the control circuitry, the energy emitting element to emit energy based on the power level.

Further disclosed herein is a catheter system that includes a balloon and control circuitry configured to control a size of the balloon by at least controlling an inflation pressure of the balloon, wherein, for example, the control circuitry can be configured to control the inflation pressure by at least controlling a flow of a fluid through an interior volume of the balloon, wherein the control circuitry may be configured to control the size of the balloon based on an input provided by a user, e.g., based on user input indicating a pressure set point or a particular balloon size, and wherein the control circuitry may be configured to control an therapeutic element based on the size of the balloon.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a schematic illustration of an example medical system including a catheter system.

FIG. 2 is a schematic illustration of the catheter system of FIG. 1 within a blood vessel of a patient.

FIG. 3 is a schematic illustration of the catheter system of FIG. 1 accessing a renal artery of a patient.

FIG. 4 is a schematic illustration of the catheter system of FIG. 1 in a delivery configuration.

FIG. 5 is a schematic illustration of the catheter system of FIG. 1 in an expanded configuration.

FIG. 6 is an example illustration depicting a pressure within and a dimension defined by a balloon of the catheter system of FIG. 1.

FIG. 7 is an example illustration depicting a parameter of a balloon and an energy level of a therapeutic element.

FIG. 8 is a flow diagram illustrating an example technique for controlling an inflation pressure of a balloon using the catheter system of FIG. 1.

FIG. 9 is a flow diagram illustrating an example technique for controlling a power level of a therapeutic element using the catheter system of FIG. 1.

### DETAILED DESCRIPTION

The present disclosure describes example catheter systems that include an elongate body configured to be positioned within a patient, at least one balloon carried by the elongate body, and at least one therapeutic element configured to deliver therapy to a target tissue site of the patient to achieve a therapeutic outcome. The catheter systems, as well as systems including the catheter systems and methods of using the catheter system, can be used for any suitable medical procedures, such as neuromodulation (e.g., denervation), that include delivering a therapy (e.g., ultrasound energy, microwave energy, and/or radiofrequency (RF) energy) to a target tissue site via the therapeutic element. The catheter system is configured to be positioned (e.g., by a clinician) within a blood vessel or other anatomical lumen of a patient. The at least one balloon is configured to be expanded within the anatomical lumen to, for example, assist in occluding the anatomical lumen, assist in maintaining the elongate body within the anatomical lumen, assist in displacing the elongate body and a wall of the anatomical lumen, assist in positioning the therapeutic element within the anatomical lumen, and/or for other reasons. For example, the catheter system may be configured such that the expanded balloon helps retain the therapeutic element in position relative to a blood vessel wall, such as by approximately centering the therapeutic element within the blood vessel.

A balloon of the catheter is configured to deliver (e.g., circulate) a fluid relative to blood vessel wall to modify a temperature at or of the target tissue site. This temperature modification can be used to provide a therapeutic outcome (e.g., cryotherapy or heat-based therapy) or can be used to help protect the vessel wall or other anatomical lumen wall. For example, to help achieve a therapeutic outcome, the therapeutic element can be configured to heat the fluid, which then creates a lesion at the target tissue site. As another example, the fluid can be configured to cool the vessel wall or the therapeutic element within the balloon or outside of the balloon to help protect the vessel wall from heat generated by the therapeutic energy delivery (e.g., by an ultrasound transducer).

While blood vessels are primarily referred to throughout the disclosure, the devices, systems, and techniques described herein are also applicable to other target tissue sites. In addition, while a single balloon catheter is primarily referred to throughout the disclosure, catheter systems described herein can include more than one balloon, such as two, three, four or more balloons.

In some examples, the balloon is configured to expand to a range of dimensions (e.g., diameters). For example, the balloon can be a compliant balloon. The balloon is configured to expand to define a particular dimension within the range based on an inflation pressure within the balloon. In examples, the expanded dimension of the balloon is selected by a clinician based on a size of the blood vessel, e.g., selected to enable the balloon to contact a wall of the blood vessel.

In some examples described herein, control circuitry of the catheter system is configured to control a dimension (also referred to herein as a size, e.g., a diameter) of the balloon by at least controlling an inflation pressure of the balloon. In this way, the control circuitry can be configured to adjust the size of the balloon to account for the size of the blood vessel in which the elongate body and the balloon are positioned. For example, in some examples, the catheter system is configured to receive an input (e.g., from a clinician) indicative of the size (e.g., a diameter) of a blood vessel in which a procedure is to be performed. Once positioned within the blood vessel, control circuitry of the catheter system is configured to inflate the balloon to a particular inflation pressure based on the input, such that the diameter of the inflated balloon enables to balloon to be in apposition with the blood vessel walls during the procedure. In some examples, control circuitry is configured to at least partially deflate the inflated balloon (e.g., based on received user input) to enable a clinician to reposition the catheter system within the patient. As used herein, a pressure of a fluid may refer to a static pressure of the fluid, a dynamic pressure of the fluid, or a total pressure of the fluid.

Hence, the catheter system is configured to cause the balloon to define one of a variety of sizes, such that the catheter system may be utilized in blood vessels of varying size. This may be advantageous in intravascular procedures in which delivery of treatment (e.g., renal neuromodulation) is required in multiple blood vessels having different vessel diameters. The catheter system enables a clinician to control the inflated size of the balloon to accommodate the differing sizes of each of the multiple vessels, such that the catheter system may be navigated to deliver treatments to each the multiple blood vessels without requiring a removal of the catheter system from the vasculature of the patient and without requiring the use of multiple catheters having expanded balloon sizes.

For example, a clinician may navigate the catheter to a first target treatment site in a first blood vessel of a patient. The clinician may deliver a first treatment to the first target treatment site by at least providing a first input causing the catheter system to inflate the balloon to accommodate a diameter of the first blood vessel. The clinician may subsequently cause a partial or full deflation of the balloon to navigate the catheter system through the vasculature to a second treatment site in a second blood vessel having a different diameter. The clinician may then cause the catheter system to inflate the balloon to accommodate the differing diameter of the second blood vessel, such that a second treatment may be delivered to the second treatment site. In like manner, the clinician may deliver treatments to additional sites in other blood vessels, such that the treatments at each the multiple blood vessels may be conducted without requiring a removal of the catheter system from the vasculature of the patient and without requiring use of two different catheters having different expanded balloon sizes.

The catheter system is configured to provide a flow path for a fluid (e.g., saline) through an interior volume of the balloon to generate the inflation pressure within the balloon. For example, the catheter system may be configured to maintain a flow of the fluid through the flow path to enable a heat transfer to or from a tissue wall to the saline (e.g., to provide cooling or heating to the tissue wall during the treatment). In some examples, the catheter system is configured to maintain a flow of fluid through the flow path to enable a heat transfer from an therapeutic element (e.g., an ultrasound element) supported within the interior volume of the balloon. The catheter system is configured to control the fluid flow in order to control the inflation pressure within the balloon, and thereby control the expanded size of the balloon within the vessel. For example, control circuitry of the catheter system can alter the fluid flow to cause a decrease in the inflation pressure within the balloon, causing a decrease in the size (e.g., diameter) of the balloon. As another example, the control circuitry can alter the fluid flow to cause an increase in the inflation pressure within the balloon, causing an increase in the size (e.g., diameter) of the balloon. In some examples, the catheter system is configured to control the fluid flow to substantially maintain (e.g., maintain or nearly maintain to the extent permitted by manufacturing tolerances) the inflation pressure within the balloon, such that the balloon defines substantially the same size (e.g., diameter).

In examples, the catheter system includes a valve configured to control the fluid flow flowing through the balloon. The valve is configured to control a flow area of a flow path for the fluid exiting the balloon to control a flow of the fluid exiting the balloon. In some examples, the valve is configured such that adjustments to the valve alter a back pressure of the fluid flow upstream of the valve. For example, adjusting the valve in a shut direction may increase the back pressure of the fluid flow upstream of the valve. Adjusting the valve in an open direction may decrease the back pressure of the fluid flow upstream of the valve. In some examples, the valve is positioned downstream of the interior volume of the balloon, such that the back pressure generated by the valve acts on the interior volume of the balloon. In examples, the catheter system is configured to control the inflation pressure within the interior volume of the balloon by at least adjusting the valve to control the back pressure generated by the fluid flow. Hence, in examples in which the inflation pressure within the balloon corresponds to a size of the balloon, the catheter system can be configured to control a flow of the fluid through the flow path in order to control a size (e.g., a diameter) of the balloon. The flow path can include, for example, an interior volume of the balloon and one or more lumens defined by an elongate body of the catheter system.

In some examples, the catheter system includes a sensor (e.g., a pressure sensor) configured to generate a pressure signal indicative of a pressure of the fluid in the flow path. The pressure of the fluid in the flow path may be indicative of the inflation pressure within the interior volume of the balloon. The catheter system may be configured such that the pressure sensed by the sensor is at least proportional to a pressure of the fluid within the interior volume of the balloon. In some examples, the control circuitry is configured to adjust a flow area of the flow path of the fluid (e.g., using the valve) to increase, decrease, and/or substantially maintain the inflation pressure within the balloon based on the pressure signal generated by the sensor. Hence, in examples in which the inflation pressure within the balloon corresponds to a size of the balloon, the catheter system can be configured to treat the pressure indicated by the pressure signal as a proxy for the size (e.g., diameter) of the balloon, to enable control circuitry to control the size of the balloon.

In some examples, the control circuitry is configured to receive information indicative of the pressure within the interior volume of the balloon, such as by receiving the pressure signal either directly or indirectly from the sensor, and cause an adjustment in the flow area of the flow path (e.g., by adjusting a position of the valve) based on the information. In some examples, the control circuitry is configured to receive an input indicative of a pressure setpoint for the interior volume of the balloon, and cause an adjustment in the flow area based on a pressure indicated by the pressure signal and the pressure setpoint, such as based on a difference between the pressure and the pressure setpoint. For example, the control circuitry may be configured to cause an adjustment in the flow area of the fluid by at least causing a position of the valve to change (e.g., to shut, to further shut, to open, and/or to further open). The positioning of the valve caused by the control circuitry may alter the back pressure generated by the fluid flow (e.g., generated upstream of the valve) and thus alter the inflation pressure within the interior volume of the balloon, causing the inflation pressure to approach and/or equal the pressure indicated by the pressure setpoint. Hence, because the inflation pressure within the balloon corresponds to and impacts a size of the balloon, the control circuitry may use the pressure sensed by the sensor as a proxy for the size (e.g., diameter) of the balloon, and use the input indicative of the pressure setpoint as a proxy for a desired size (e.g., a desired diameter) of the balloon, and thereby control the flow path of the fluid such that the balloon achieves and/or substantially maintains the desired size.

In addition to or instead of the controlling a flow area of the fluid used to inflate the balloon based on a pressure setpoint, in some examples, the control circuitry is configured to control an energy level of the therapeutic element based on a size (e.g., a diameter) of the balloon. In some examples, the control circuitry is configured to control the flow area of the fluid based on a power level setting of the therapeutic element. The catheter system may be configured such that a displacement between the therapeutic element and a vessel wall is proportional to and/or dependent on the size of an inflated balloon within the blood vessel. For example, the therapeutic element can be positioned within the interior volume of the balloon. Due to this separation between the therapeutic element and the blood vessel wall, for larger vessels, more energy may need to be delivered via the therapeutic element to achieve a desired therapeutic outcome compared to smaller blood vessels. For example, a larger separation between the therapeutic element and the blood vessel wall (e.g., corresponding to a larger blood vessel) may increase the area over which energy from the therapeutic element is distributed, such that an increase in the energy emitted by the therapeutic element may be necessary in order to provide a sufficient amount of energy to the vessel wall. Thus, in some examples, the control circuitry is configured to determine a size (e.g., a diameter) and/or inflation pressure of the inflated balloon and determine a power level for the therapeutic element based on the size of the inflated balloon. In examples, the control circuitry is further configured to determine the power level based on a type of media (e.g., saline inflating the balloon) through which the therapeutic element transmits energy over the resulting displacement, because different media may heat differently or otherwise transfer thermal energy differently.

In some examples, the control circuitry is configured to control inflation of the balloon based on an assessment that the inflating balloon has expanded sufficiently to contact a vessel wall. For example, the balloon may be configured such that, as the balloon expands within a blood vessel in a substantially unconstrained state (e.g., prior to contacting the vessel wall), the inflation pressure within the interior volume of the balloon defines an increasing trend profile. In some examples, the control circuitry determines the trend profile using a pressure signal from a sensor configured to sense the inflation pressure. In examples, the control circuitry is configured to identify a departure from the trend profile (e.g., identify a relatively sudden, steeper increase in the interior pressure, or the like) indicating that the balloon may have increased in size sufficiently such that the vessel wall is a least partially resisting a continued expansion of the balloon. The control circuitry may be configured to alter the flow rate of the fluid through the balloon in response to the departure from the trend profile. For example the control circuitry can be configured to alter the flow rate such that the inflation pressure within the balloon substantially ceases increasing, thereby enabling the size of the balloon to remain relatively stable once the balloon expands to contact the vessel wall.

The therapeutic element described herein can be used to provide any suitable type of therapy, such as denervation therapy. Conditions such as arrhythmias, hypertension, states of volume overload (e.g., heart failure), and progressive renal disease due to excessive activation of the sympathetic nervous system (SNS), may be mitigated by modulating the activity of overactive nerves (neuromodulating), for example, by denervating or reducing the activity of the overactive nerves. Some sympathetic nerves, such as sympathetic nerves of the kidneys, are positioned proximate to blood vessels, such that these overactive nerves may be chemically, thermally, or electrically denervated by ablating sympathetic nerve tissue in or near the blood vessels (e.g., renal blood vessels). A chemical, a thermal energy (e.g., heat energy or cryotherapeutic energy), or an electrical energy may be delivered to and/or generated within the sympathetic tissue via a therapeutic element carried by a catheter and positioned within the vasculature of a patient.

In neuromodulation, one or more therapeutic elements may be introduced near one or more target nerves. In renal neuromodulation, for example, the one or more therapeutic elements may be introduced near renal nerves located between an aorta and a kidney of a patient. In some examples, the one or more therapeutic elements may be carried by or attached to a catheter, and the catheter may be introduced intravascularly, e.g., into a renal artery via a brachial artery, femoral artery, or radial artery approach. In other examples, the one or more therapeutic elements may be introduced extravascularly, e.g., using a laparoscopic technique.

Although the present technology is herein described in many instances with reference to renal nerves and vessels, the present technology also has application to neuromodulation at other anatomical sites (e.g., spinal neuromodulation, cardiac neuromodulation, brain neuromodulation, sacral neuromodulation, urinary neuromodulation, and/or neuromodulation techniques directed to other portions of a body) and their associated nerves and that such devices and systems can be configured (e.g., have suitable shape and dimensions) for such sites. For example, a catheter may be configured to deliver energy with a portion of the catheter carrying a therapeutic element positioned with a particular anatomical lumen or a particular tissue (e.g., a renal artery, external iliac artery, internal iliac artery, internal pudendal artery, celiac artery, mesenteric artery, superior mesenteric artery, inferior mesenteric artery, hepatic artery, splenic artery, gastric artery, left gastric artery, pancreatic artery, uterine artery, ovarian artery, testicular artery, and/or their associated arterial branches, accessories, veins, and/or other hollow anatomical structures).

As used herein, the terms “distal” and proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction toward the clinician or clinician's control device.

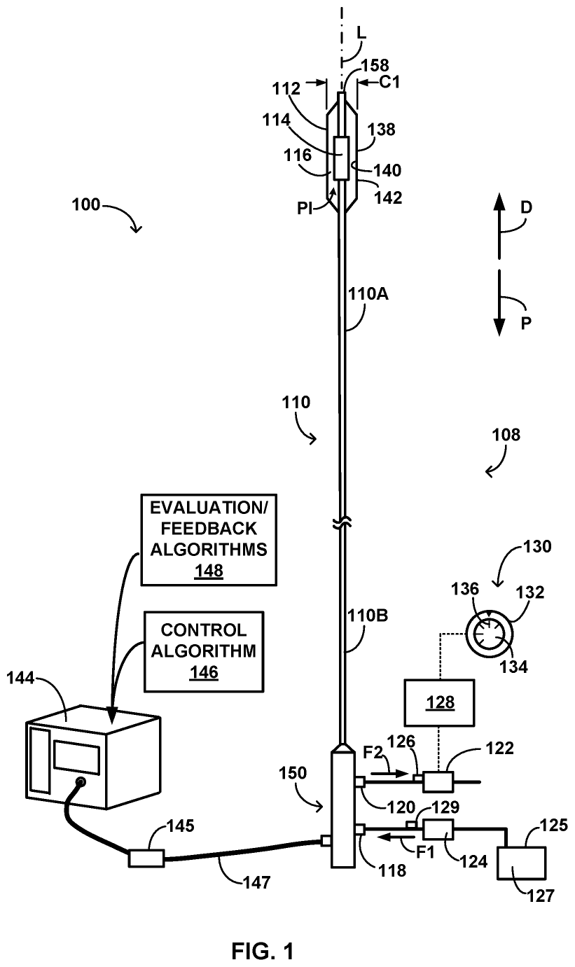

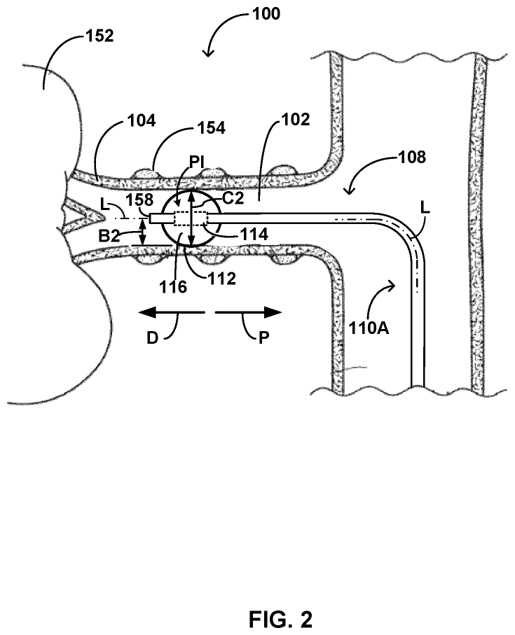

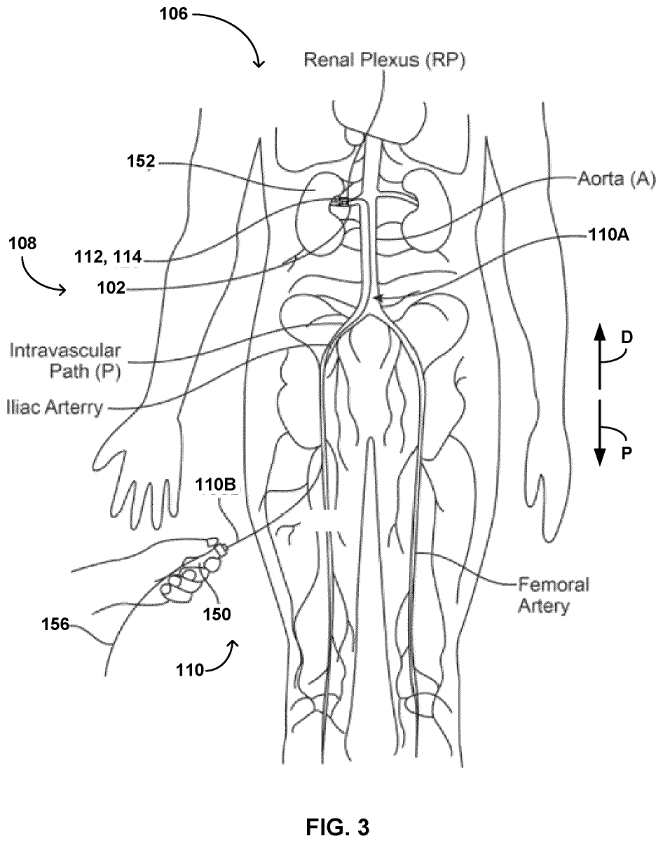

FIG. 1 is a partially schematic perspective view illustrating a medical system 100 configured in accordance with examples of the present disclosure. FIG. 2 is a schematic illustration of a portion of medical system 100 within a blood vessel 102 of a patient 106, the blood vessel 102 having a vessel wall 104. FIG. 3 illustrates medical system 100 being navigated through vasculature of a patient 106 to a target treatment site within blood vessel 102. In the examples of FIGS. 2 and 3, blood vessel 102 is a renal artery and vessel wall 104 is a renal artery wall. However, in other examples, medical system 100 is be configured to deliver treatments to other blood vessels, anatomical lumens, and/or other tissues, such as an external iliac artery, internal iliac artery, internal pudendal artery, celiac artery, mesenteric artery, superior mesenteric artery, inferior mesenteric artery, hepatic artery, splenic artery, gastric artery, left gastric artery, pancreatic artery, uterine artery, ovarian artery, testicular artery, and/or their associated arterial branches, accessories, veins, and/or other hollow anatomical structures of a patient.

Medical system 100 includes a catheter system 108 defining an elongate body 110 configured to be positioned (e.g., by a clinician) within blood vessel 102 of patient 106. Catheter system 108 includes one or more expandable elements such as balloon 112 (“balloon 112”) configured to expand (e.g., by inflation) when elongate body 110 is positioned within blood vessel 102. Balloon 112 may be configured to expand to, for example, assist in occluding blood vessel 102 during a procedure, assist in maintaining elongate body 110 within blood vessel 102, assist in displacing and/or maintaining a displacement between elongate body 110 and vessel wall 104, and/or for other reasons. Elongate body 110 defines a longitudinal axis L. Balloon 112 may be configured to expand radially outwards relative to longitudinal axis L (e.g., substantially perpendicular to longitudinal axis L) when balloon 112 is inflated within blood vessel 102 of patient 106.

Catheter system 108 includes an therapeutic element 114 configured to deliver a therapy to tissue to, for example, conduct a neuromodulation or another procedure on vessel wall 104 and/or other tissues associated with blood vessel 102. While the therapy is primarily referred to herein as energy (e.g., acoustic, radiofrequency, pulsed field, thermal, or direct electrical current), the therapy can be in the form of any suitable modality in other examples.

Balloon 112 is configured to help position therapeutic element 114 within blood vessel 102 during the procedure. For example, catheter system 108 may be configured such that inflation of balloon 112 helps retain therapeutic element 114 in a position (e.g., approximately centered in blood vessel 102) relative to vessel wall 104, helps maintain a displacement between therapeutic element 114 and vessel wall 104, and/or assists in other ways. In some examples, elongate body 110 supports (e.g., mechanically supports) balloon 112. Elongate body 110 may also support (e.g., mechanically supports) therapeutic element 114. For example, therapeutic element 114 can be positioned within an interior volume 116 of balloon 112 or external to balloon 112, such as on an outer surface of balloon 112.

Catheter system 108 is configured to control an inflation pressure PI within an interior volume 116 of balloon 112 to control a size (e.g., a diameter) of balloon 112. Catheter system 108 is configured to control the inflation pressure PI by at least controlling the pressure of a flow of a fluid (e.g., saline) within interior volume 116. In some examples, catheter system 108 is configured to enable a substantially continuous (e.g., continuous or nearly continuous) flow of fluid flowing through interior volume 116 to achieve the desired inflation pressure PI. Catheter system 108 may be configured to adjust and/or establish the flow rate (e.g., a mass flow) through interior volume 116 in order to adjust and/or establish the inflation pressure PI within interior volume 116, and thus adjust and/or establish a size of balloon 112.

For example, catheter system 108 may be configured such that the fluid may flow into a fluid inlet 118 (e.g., flow F1), through an inlet lumen defined by elongate body 110, through interior volume 116, then through an outlet lumen defined by elongate body 110, and subsequently discharge through a fluid outlet 120 (e.g., flow F2). Thus, the inlet lumen, interior volume 116, and the outlet lumen can define a flow path for fluid that flows through catheter system 108. Catheter system 108 includes a valve 122 configured to control a flow area (e.g., by throttling the fluid flow) of the fluid. The flow area may be, for example, a flow area defined by a valve 122 (e.g., defined by a position of a restricting element of valve 122 relative to a seat of valve 122). The position of valve 122 may thus control a pressure of the fluid flow at least within some portion of the flow path (e.g., a portion upstream of valve 122). Catheter system 108 may be configured such that the pressure within the portion of the flow path influences the inflation pressure PI within interior volume 116, such that the inflation pressure PI of balloon 112 may be adjusted and/or established by the position of valve 122. Hence, catheter system 108 is configured such that a position of valve 122 controls a size of balloon 112.

In examples, catheter system 108 includes a pump 124 configured to drive the fluid flow through the flow path defined by catheter system 108. Catheter system 108 may include a fluid container 125 defining a reservoir 127 configured to hold a volume of the fluid. Pump 124 may be configured to draw the fluid from reservoir 127 and discharge the fluid into the flow path. In some examples, pump 124 is a positive displacement pump, such as a peristaltic pump, a diaphragm pump, a lobe pump, a piston pump, a screw pump, a gear pump, a rotary vane pump, or other positive displacement pump. In examples, pump 124 is a dynamic pump, such as a centrifugal pump, a cantilever pump, a vertical centrifugal pump, a multistage centrifugal pump, or other dynamic pump. In examples, pump 124 is an axial-flow pump or a radial flow pump.

In some examples, valve 122 is located in a portion of the flow path downstream of interior volume 116 of balloon 112. Valve 122 may be configured such that adjusting valve 122 in a shut direction increases a pressure of the fluid flow upstream of valve 122 (e.g., increases a back pressure), causing an increase in the inflation pressure PI within interior volume 116, and such that adjusting valve 122 in an open direction decreases a pressure of the fluid flow upstream of valve 122 (e.g., decreases the back pressure), causing a decrease in inflation pressure PI within interior volume 116. Valve 122 is configured such that when the position of valve 122 is substantially unchanging (e.g., unchanging or unchanging to the extent permitted by manufacturing tolerances), the pressure of the fluid flow upstream of valve 122 (e.g., the back pressure) and/or the inflation pressure PI within interior volume 116 are substantially unchanging. Hence, when a size of balloon 112 is a function of the inflation pressure PI within interior volume 116, catheter system 108 may be configured to use valve 122 to control the inflation pressure PI within balloon 112 in order to control a size (e.g., a diameter) of balloon 112.

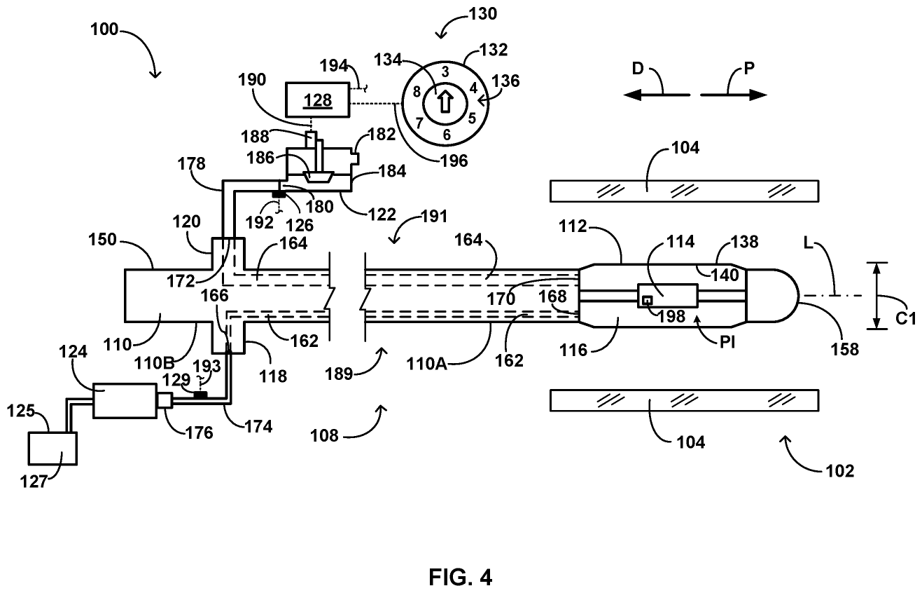

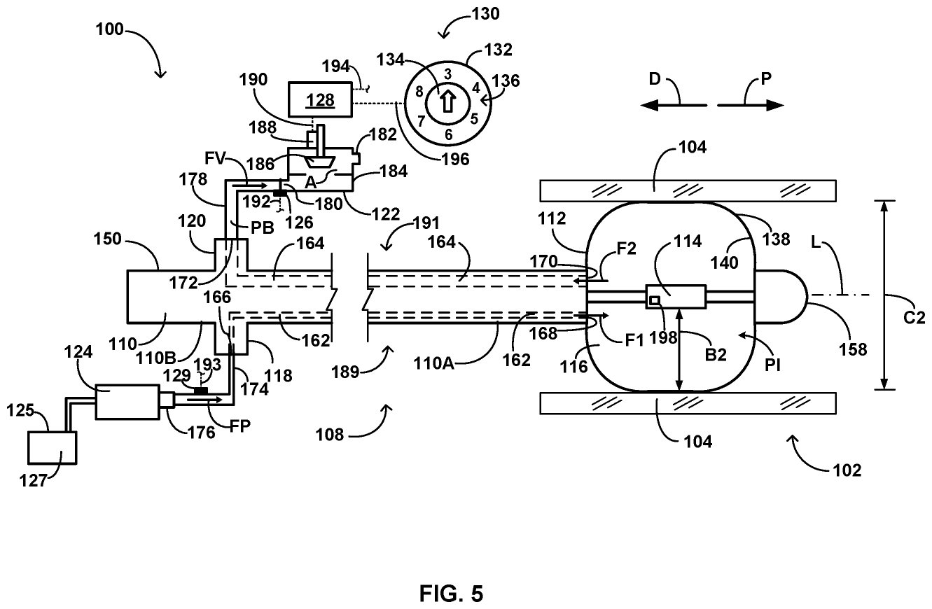

In some examples, catheter system 108 includes a sensor 126 configured to sense a parameter indicative of the inflation pressure PI of balloon 112, and generate a pressure signal indicative of the inflation pressure PI. For example, sensor 126 can include a pressure sensor configured to generate a pressure signal indicative of a pressure within interior volume 116 or at another location along the flow path described above, such as in a lumen of elongate body 110 or another location. In examples, the parameter indicative of the inflation pressure PI sensed by sensor 126 is a pressure upstream of a flow area of valve 122 (e.g., flow area A (FIG. 4, FIG. 5)). Catheter system 108 can be configured to adjust a position of valve 122 to increase, decrease, and/or maintain the inflation pressure PI within balloon 112 based on the pressure signal generated by sensor 126.

Catheter system 108 may include control circuitry 128 configured to control an inflation pressure PI of balloon 112. In some examples, control circuitry 128 is configured to receive the pressure signal and cause an adjustment in the inflation pressure PI within balloon 112 based on the pressure signal. For example, control circuitry 128 can be configured to receive an input indicative of a pressure setpoint, and cause an adjustment in the position of valve 122 based on a pressure indicated by the pressure signal and the pressure setpoint, such as based on a difference between the pressure and the pressure setpoint. Hence, with a size of the balloon corresponding to the inflation pressure PI within interior volume 116 of the balloon 112, control circuitry 128 may treat the pressure sensed by sensor 126 as a proxy for the size (e.g., diameter) of balloon 112, treat the input indicative of the pressure setpoint as a proxy for a desired size (e.g., a desired diameter) of balloon 112, and control the position of valve 122 such that balloon 112 achieves and/or substantially maintains the desired size.

In examples, catheter system 108 may be configured to substantially determine a flow rate through interior volume 116 of balloon 112. For example, catheter system 108 may include a flow sensor 129 configured to sense a parameter indicative of a flow rate through interior volume 116 of balloon 112. Catheter system 108 may be configured to substantially determine the flow rate through interior volume 116 based on a speed of pump 124, or using another indication. Control circuitry 128 may be configured to adjust one or more of the position of valve 122 or a flow rate provided by pump 124 to control the inflation pressure PI. In examples, control circuitry 128 is configured to adjust one or more of the position of valve 122 or the flow rate provided by pump 124 to maintain the flow rate through interior volume 116 above a flow threshold, such as above 5 ml/min. In some examples, control circuitry 128 is configured to optimize (e.g., substantially maximize) the flow rate through interior volume 116 for a given inflation pressure PI within interior volume 116 (e.g., based on the pressure signal from sensor 126).

Control circuitry 128, as well as other processors, processing circuitry, controllers, control circuitry, and the like, described herein, may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, control circuitry 128 includes multiple components, such as any combination of one or more microprocessors, one or more DSPs, one or more ASICs, or one or more FPGAs, as well as other discrete or integrated logic circuitry, and/or analog circuitry.

Although not shown in FIG. 1, system 100 can also include memory 44 configured to store program instructions, such as software, which may include one or more program modules, which are executable by control circuitry 128. When executed by control circuitry 128, such program instructions may cause control circuitry 128 to provide the functionality ascribed to control circuitry 128 herein. The program instructions may be embodied in software and/or firmware. The memory can include any volatile, non-volatile, magnetic, optical, or electrical media, such as a random access memory (RAM), read-only memory (ROM), non-volatile RAM (NVRAM), electrically-erasable programmable ROM (EEPROM), ferroelectric RAM (FRAM), flash memory, or any other digital media.

In some examples, catheter system 108 includes a user interface 130 configured to receive the input indicative of the pressure setpoint from a user and provide the input to control circuitry 128. User interface 130 may be configured to receive the indicative input from a clinician and/or other user, such that the clinician and/or other user may specify a desired size for balloon 112. User interface 130 can have any suitable configuration sufficient to receive an input from a user. For example, user interface 130 can include a button or keypad, a touch screen, a speaker configured to receive voice commands from a user, and/or a display, such as a liquid crystal (LCD), light-emitting diode (LED), or organic light-emitting diode (OLED). In some examples, user interface 130 includes a device body 132 and a positioning member 134 configured to move (e.g., rotate) relative to device body 132 to define a desired size of balloon 112. In these examples, user interface 130 may include one or more indicia 136 indicative of the position of positioning member 134 relative to device body 132, such that a clinician may place positioning member 134 in a particular position relative to device body 132 to specify the desired size for balloon 112. In some examples, user interface 130 is configured to display information, such as one or more desired sizes and/or setpoints (e.g., the current desired size and/or setpoint being used by control circuitry 128 or one or more predetermined desired sizes and/or setpoints from which the user can select to input a desired size of balloon 112).

In some examples, elongate body 110 is configured to support therapeutic element 114 at a fixed longitudinal location (measured along longitudinal axis L) on elongate body 110 relative to balloon 112. For example, as illustrated in FIG. 1 and FIG. 2, elongate body 110 may support therapeutic element 114 such that therapeutic element 114 is positioned within an interior volume 116 of balloon 112. In examples, elongate body 110 is configured to support therapeutic element 114 such that balloon 112 substantially extends over (e.g., substantially surrounds) therapeutic element 114. In some examples, elongate body 110 supports therapeutic element 114 such that therapeutic element 114 is positioned distal to balloon 112 (e.g., displaced from balloon 112 in the direction D) or proximal to balloon 112 (e.g., displaced from balloon 112 in the direction P). In some examples, balloon 112 supports therapeutic element 114, such that, for example, inflation of balloon 112 decreases a displacement between therapeutic element 114 and vessel wall 104. For example, therapeutic element 114 may be supported by an exterior surface 138 of balloon 112 (“balloon exterior surface 138”), an interior surface 140 of balloon 112 (“balloon interior surface 140”), and/or some portion of a balloon body 142 between and/or defining balloon exterior surface 138 and/or balloon interior surface 140.

Elongate body 110 defines a distal portion 110A (“distal body portion 110A”) and a proximal portion 110B (“proximal body portion 110B”). Therapeutic element 114 and/or balloon 112 are positioned on distal portion 110A in the example shown in FIG. 1. In examples, catheter system 108 is configured to assume a relatively low profile delivery configuration in which at least one of distal portion 110A and/or balloon 112 defines a dimension C1 (e.g., a diameter), which can be measured in a direction perpendicular to longitudinal axis L. The dimension C1 may define a displacement sufficient to allow the passage of at least distal body portion 110A and balloon 112 through vasculature of patient 106 to reach a target treatment site within patient 106. In some examples, distal body portion 110A is configured to locate therapeutic element 114 at an intraluminal (e.g., intravascular) location. In examples, catheter system 108 is configured such that, in the delivery configuration, the dimension C1 measures 2, 3, 4, 5, 6, or 7 French or another suitable size. Balloon 112 is configured to expand from the delivery configuration to an expanded configuration (FIG. 2) to, for example, position and/or stabilize distal body portion 110A and/or therapeutic element 114 when distal body portion 110A locates therapeutic element 114 at the target treatment site.

Medical system 100 may include a generator 144 is configured to control, monitor, supply, and/or otherwise support operation of catheter system 108. In other examples, catheter system 108 may be self-contained and/or otherwise configured for operation independent of generator 144. When present, generator 144 can be configured to generate a selected form and/or magnitude of energy for delivery to tissue at a treatment site via catheter system 108 (e.g., therapeutic element 114). For example, generator 144 can be configured to generate energy (e.g., pulsed field, electrical current, microwave, radiofrequency, monopolar, and/or bipolar energy). In other examples, generator 144 may be another type of device configured to generate and deliver another suitable type of energy to catheter system 108.

Medical system 100 includes a cable 147 configured to deliver power from generator 144 and catheter system 108. Along cable 147 or at another suitable location within medical system 100, medical system 100 may include a control device 145 configured to initiate, terminate, and/or adjust operation of one or more components of catheter system 108 directly and/or via generator 144. In some examples, generator 144 may be configured to execute an automated control algorithm 146 and/or to receive control instructions from an operator. Similarly, in some implementations, generator 144 is configured to provide feedback to an operator before, during, and/or after a treatment procedure via an evaluation/feedback algorithm 148. In examples, control circuitry 128 is configured to execute automated control algorithm 146 and/or evaluation/feedback algorithm 148, and/or communicate with processing circuitry within generator 144 to cause execution of automated control algorithm 146 and/or evaluation/feedback algorithm 148.

In some examples, catheter system 108 is configured to receive energy (e.g., from generator 144) and convert the energy (e.g., electrical current) into acoustic energy (e.g., sound pressure waves). For example, therapeutic element 114 can be include an ultrasound transducer configured to transmit the acoustic energy to vessel wall 104 or another anatomical location of patient 106. In examples, therapeutic element 114 is configured to transmit acoustic energy in a frequency range of from about 20 kilohertz (kHz) to about 200 megahertz (MHz). Catheter system 108 (e.g., therapeutic element 114) may be configured such that the acoustic energy tends to induce molecular vibration and friction in tissues at a target site at an anatomical location of patient 106, resulting in absorptive heating within the target tissues. In examples, catheter system 108 may be configured such that the acoustic energy tends to heat a fluid within interior volume 116 of balloon 112 to transfer thermal energy to tissues at the target site. System 100 is configured to control the power and/or wavelength of the acoustic energy transmitted from therapeutic element 114 to, for example, control a temperature of the tissues at the target site receiving the acoustic energy and/or thermal energy.

In examples, catheter system 108 is configured to inflate balloon 112 with a fluid such as saline. Catheter system 108 may be configured such that the fluid serves as a transmission media for acoustic energy transmitted from therapeutic element 114 to the target tissue.

In some examples, medical system 100 (e.g., proximal body portion 110B) includes a handle portion 150, which is configured to remain outside vasculature of a patient when distal body portion 110A is within vasculature of the patient. Handle portion 150 may be configured to allow a clinician to navigate at least distal body portion 110A through the vasculature, allow inflation and/or deflation of balloon 112, allow the transmission and/or control of energy delivered to therapeutic element 114, and/or enable other functions of medical system 100 which may assist in the delivery of a treatment (e.g., a neuromodulation) to patient 106. At least some portion of catheter system 108 (e.g., distal body portion 110A) may be substantially flexible, such that catheter system 108 may flex and/or bend enroute to positioning therapeutic element 114 substantially at a target location within a blood vessel of a patient. Hence, although illustrated as substantially linear in FIG. 1, catheter system 108 (or portions thereof) may be configured to assume linear, curved, and/or curvilinear shapes. Correspondingly, longitudinal axis L (and/or portions thereof) defined by catheter system 108 may be linear, curved, and/or curvilinear.

FIG. 2 schematic illustrates a portion of medical system 100 (e.g., distal body portion 110A, balloon 112, and therapeutic element 114) within blood vessel 102 defined by vessel wall 104 of patient 106. Catheter system 108 is configured to enable inflation of balloon 112 to cause catheter system 108 to transition from the delivery configuration (FIG. 1) to an expanded configuration when distal body portion 104A positions balloon 112 and/or therapeutic element 114 within blood vessel 102. In examples, catheter system 108 is configured to expand balloon 112 such that balloon 112 defines a dimension C2 (e.g., a diameter) in an expanded configuration. The dimension C2 defined in the expanded configuration may be greater than the dimension C1 defined in the delivery configuration (FIG. 1). In examples, balloon 112 is configured to define the dimension C1, the dimension C2, and/or another dimension (e.g., a dimension substantially perpendicular to longitudinal axis L) based on an inflation pressure PI of a fluid within interior volume 116.

Catheter system 108 is configured to control the inflation pressure PI such that the dimension C2 may be adjusted to accommodate the size of blood vessel 102. For example, catheter system 108 may be configured to cause balloon 112 to expand such that balloon 112 (e.g., balloon exterior surface 138) contacts vessel wall 104. Distal body portion 110A may support balloon 112 such that, when balloon 112 defines the dimension C2, catheter system 108 defines a displacement B2 substantially between therapeutic element 114 and balloon exterior surface 138. Displacement B2 can, for example, define a displacement between therapeutic element 114 and vessel wall 104 when balloon exterior surface 138 contacts vessel wall 104. In examples, catheter system 108 is configured to substantially center (e.g., center or nearly center to the extent permitted by vessel symmetry) therapeutic element 114 within blood vessel 102 when balloon 112 expands to contact vessel wall 104.

Therapeutic element 114 (FIG. 1) may transmit energy and/or cause transmission of thermal energy to tissue of patient 106 to induce one or more desired effects (e.g., neuromodulation effects) on localized regions of blood vessel 102 and regions adjacent to blood vessel 102. For example, when blood vessel 102 defines a renal artery associated with a kidney 152, therapeutic element 114 may induce one or more desired neuromodulating effects to a portion of Renal Plexus (RP) 154 lying within or adjacent to the adventitia of the renal artery.

FIG. 3 illustrates a portion of medical system 100 positioned within vasculature of a patient 106 to place therapeutic element 114 within blood vessel 102. FIG. 3 is described with primary reference to a renal artery, however similar devices, systems, and techniques may be adapted for accessing other anatomical lumens or tissues within patient 106.

Catheter system 108 (e.g., elongate body 110) may provide access to the renal plexus (RP) through an intravascular path (P), such as a percutaneous access site in the femoral (illustrated), brachial, radial, or axillary artery to a target treatment site within blood vessel 102. By manipulating proximal body portion 110B of elongate member 110 from outside the intravascular path (P), a clinician may advance at least distal body portion 110A through the sometimes-tortuous intravascular path (P) and remotely manipulate distal body portion 110A. In examples, distal body portion 110A may be remotely manipulated by a clinician using handle portion 150.

In the example illustrated in FIG. 3, balloon 112 is delivered intravascularly to the treatment site using a guidewire 156 in an OTW technique. Catheter system 108 (e.g., elongate body 110) may define a passageway for receiving guidewire 156 for delivery of elongate body 110 (e.g., distal body portion 110A) using either an OTW or a RX technique. At the treatment site, guidewire 156 can be at least partially withdrawn or removed, and balloon 112 may be expanded from the delivery configuration (FIG. 1) to an expanded configuration (FIG. 2). Balloon 112 may substantially position therapeutic element 114 relative to blood vessel 102 for delivering energy to blood vessel 102 and/or other anatomical lumens or tissues within patient 106. In other examples, balloon 112 and/or therapeutic element 114 may be delivered to the treatment site within a different guide device, such as guide sheath (not shown in FIG. 3), with or without using guidewire 156. In examples in which medical system 100 includes a guide sheath, when balloon 112 and/or therapeutic element 114 are at the target treatment site, the guide sheath may be at least partially withdrawn or retracted and balloon 112 may be transformed into an expanded configuration. In still other examples, elongate body 110 may be steerable itself such that balloon 112 and/or therapeutic element 114 may be delivered to the treatment site without the aid of guidewire 156 and/or a guide sheath.

FIG. 4 is a schematic illustration of medical system 100 with catheter system 108 in a delivery configuration within blood vessel 102, with catheter system 108 defining a dimension C1. FIG. 5 is a schematic illustration of medical system 100 within catheter system 108 in an expanded configuration within blood vessel 102, with catheter system 108 defining a dimension C2. Dimension C2 is greater than dimension C1. In examples, as shown in FIG. 5, catheter system 108 is configured to cause balloon 112 to contact vessel wall 104 in the expanded configuration. Dimension C1 and/or the dimension C2 may be a cross-sectional dimension of catheter system 108 (e.g., balloon 112), the cross-section being taken perpendicular to longitudinal axis L of elongate body 110. In examples, dimension C1 and dimension C2 define a displacement in a direction substantially perpendicular to longitudinal axis L. In examples, longitudinal axis L extends through catheter system 108 through a distal end 158 of elongate body 110 (“body distal end 158”). Longitudinal axis L may extend through at least some portion of distal body portion 110A and/or proximal body portion 110B.

Catheter system 108 is configured to control a dimension defined by balloon 112 by at least controlling the inflation pressure PI in interior volume 116. Catheter system 108 is configured to generate the inflation pressure PI using a fluid flowing through interior volume 116 of balloon 112. In examples, catheter system 108 is configured such that the fluid flows from fluid inlet 118 to interior volume 116 and from interior volume 116 to fluid outlet 120. Catheter system 108 may control the inflation pressure PI by at least adjusting a flow area A (FIG. 5) defined by valve 122 through which the fluid flows and/or adjusting a flow rate provided by pump 124. In examples, catheter system 108 controls the inflation pressure PI (e.g., by adjusting the flow area A) based on a pressure signal provided by sensor 126.

In some examples, control circuitry 128 is configured to receive the pressure signal and position valve 122 (e.g., to adjust the flow area A) and/or adjust the flow rate provided by pump 124 based on the pressure signal. For example, control circuitry 128 may receive an input (e.g., from a clinician) via user interface 130 indicative of a desired size (e.g., a diameter) of a blood vessel in which a procedure is to be performed. Hence, with a size of balloon 112 corresponding to the inflation pressure PI within interior volume 116, control circuitry 128 may treat the pressure sensed by sensor 126 as a proxy for the size (e.g., diameter) of balloon 112, treat the input indicative of the pressure setpoint as a proxy for the desired size, and control the position of valve 122 such that balloon 112 achieves and/or substantially maintains the desired size.

Dimension C1 and/or dimension C2 may define any suitable displacements. Further, catheter system 108 is configured such that dimension C1 and/or dimension C1 may define any one displacement over a range of possible displacements. For example, catheter system 108 may be configured such that balloon 112 and/or elongate body 110 define a minimum cross-sectional dimension (e.g., a minimum diameter) and a maximum cross-sectional dimension (e.g., a maximum diameter) depending on, for example, an inflation pressure PI within interior volume 116. Hence, although illustrated for clarity in FIGS. 4 and 5 as corresponding to only one displacement value, catheter system 108 is configured such that dimension C1 and dimension C2 may define any displacement greater than or equal to the minimum cross-sectional dimension and less than or equal to the maximum cross-sectional dimension.

Balloon 112 is configured to expand radially outward in a direction away from longitudinal axis L of elongate body 110 when an inflation pressure PI sufficient to cause the expansion acts within interior volume 116. In examples, interior volume 116 is bound at least in part by balloon interior surface 140. Balloon 112 (e.g., balloon body 142) may be configured to elastically expand based on the inflation pressure PI within interior volume 116, such that an increase in the inflation pressure PI causes balloon 112 to increase its cross-sectional diameter (e.g., from dimension C1 to dimension C2) and a decrease in the inflation pressure PI causes balloon 112 to decrease its cross-sectional dimension (e.g., from dimension C2 to the dimension C1). Thus, medical system 100 is configured such that catheter system 108 may define a first dimension (e.g., dimension C1) when catheter system 108 is navigated (e.g., by a clinician) to and/or from a target site within blood vessel 102, and define a second dimension greater than the first dimension (e.g., dimension C2) while delivering a treatment (e.g., a neuromodulation) at the target site.

In examples, balloon 112 is configured to expand radially to define a specific dimension based on the inflation pressure PI within interior volume 116. Balloon 112 may be configured such that the specific dimension increases as the specific inflation pressure PI increases. In examples, the specific dimension is a function of the inflation pressure PI. Catheter system 108 (e.g., control circuitry 128) may be configured to define the specific dimension of balloon 112 based on a pressure indicative of the inflation pressure PI, such that catheter system 108 may use the inflation pressure PI as a proxy for the size (e.g., diameter) of balloon 112. The specific dimension for a given inflation pressure may be a singular value for the given inflation pressure, or may be a value defined within a range of values for the given inflation pressure. For example, the specific value for the specific inflation pressure may be the minimum cross-sectional dimension of balloon 112, the maximum cross-sectional dimension of balloon 112, or a value between the minimum cross-sectional dimension and the maximum cross-sectional dimension.

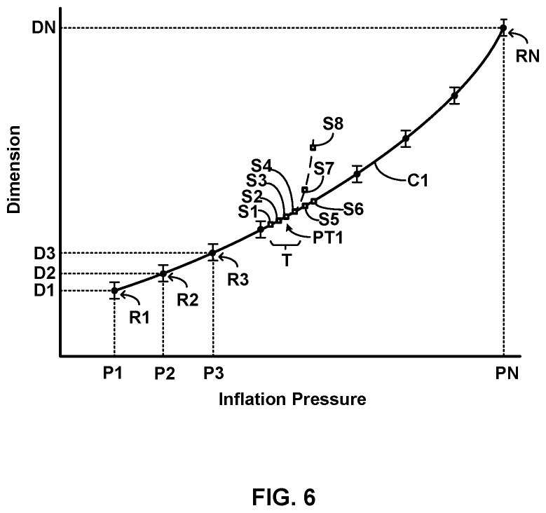

As an example, FIG. 6 illustrates example relationships between specific values defined by balloon 112 and specific inflation pressures within interior volume 116 of balloon 112. In this example, balloon 112 is configured to define one or more specific dimensions such as D1, D2, D3, . . . , DN when catheter system 108 causes an specific pressure such as P1, P2, P3, . . . , PN, respectively, within interior volume 116 (e.g., when the inflation pressure PI is one of P1, P2, P3, . . . , PN). For example, balloon 112 may be configured such that specific inflation pressure P1 causes balloon 112 to define specific dimension D1, specific inflation pressure P2 causes balloon 112 to define specific dimension D2, specific inflation pressure P3 causes balloon 112 to define specific dimension D3, specific inflation pressure PN causes balloon 112 to define specific dimension DN, and so on. In examples, specific dimension D1 is a dimension within a range R1, specific dimension D2 is a dimension within a range R2, specific dimension D3 is a dimension within a range R3, and/or specific dimension DN is a dimension with a range RN.

In examples, the specific dimensions D1, D2, D3, . . . , DN increase as the specific inflation pressures P1, P2, P3, . . . , PN increase. In some examples, the specific dimensions D1, D2, D3, . . . , DN are described by a correlation C1 relating a specific dimension to a specific inflation pressure. In some examples, the correlation C1 is continuous, such that correlation C1 describes a specific dimension for any specific pressure between and including specific pressure P1 and specific pressure PN. The correlation C1 may be a discrete, such that such that correlation C1 describes a specific dimension when the specific pressure defines or is assigned (e.g., by control circuitry 128) a discrete value (e.g., one or P1, P2, P3, . . . , PN). In some examples, the correlation C1 is defined by a coordinate data structure (e.g., a table) describing a plurality of ordered pairs describing a set of coordinates (e.g., a coordinate describing a specific inflation pressure and a coordinate describing a specific dimension).

In some examples, the correlation C1 is defined by a plurality of separate correlations defined over one or more intervals, such as an interval between P1 and P2, an interval between P2 and P3, and/or another interval. The correlation C1 may define any curvature and/or slope over any interval, and may be a linear function over one or more intervals. Catheter system 108 may be configured to cause the inflation pressure PI to substantially achieve a specific inflation pressure (e.g., one of P1, P2, P3, . . . , PN) within interior volume 116 in order to cause balloon 112 to define a specific dimension (e.g., one of D1, D2, D3, . . . , DN) associated with the specific inflation pressure.

The one or more specific dimensions such as D1, D2, D3, . . . , DN may be a cross-sectional dimension of balloon 112, such as dimensions substantially perpendicular to longitudinal axis L (e.g., perpendicular or nearly perpendicular to extent permitted by manufacturing tolerances). The one or more specific dimensions such as D1, D2, D3, . . . , DN may be one of dimension C1 (FIG. 4), dimension C2 (FIG. 5), or a dimension other than dimension C1 or dimension C2. The one or more specific dimensions such as D1, D2, D3, . . . , DN may define of the minimum cross-sectional dimension of balloon 112, the maximum cross-sectional dimension of balloon 112, or a cross-sectional dimension other than the minimum cross-sectional dimension and the maximum cross-sectional dimension.

Balloon 112 has any suitable configuration. In examples, balloon 112 is configured to such that an imaging system (e.g., an imaging system extracorporeal to patient 106) can capture an image of balloon 112 when balloon 112 is within patient 106. In examples, balloon 112 (e.g., balloon body 142) comprises silicone. In some examples, balloon 112 is a dipped balloon fabricated using a dip molding process. Balloon 112 (e.g., balloon body 142) may be constructed of any compliant, semi-compliant or non-compliant material, typically a plastic such as polyurethane, nylon, polyethylene, PET or PEBAX.

Catheter system 108 is configured to provide a flow path for a fluid (e.g., saline) through interior volume 116 of balloon 112. Catheter system 108 is configured such that the fluid flowing through interior volume 116 exerts the inflation pressure PI on balloon interior surface 140 as the fluid flows through interior volume 116. Catheter system 108 may be configured to control the flow of the fluid to control the inflation pressure PI, and thereby control the size of balloon 112. For example, catheter system 108 may provide the flow of fluid to cause an inflation pressure PI such as one of the specific pressures P1, P2, P3, . . . , PN, such that balloon 112 defines on of the corresponding dimensions D1, D2, D3, . . . , DN, respectively (FIG. 6). Control circuitry 128 can control the inflation pressure PI by at least adjusting the flow area A (FIG. 5). Further, and as discussed, the flow of the fluid through the flow path can provide one or more other functions, such as facilitating a heat transfer to or from vessel wall 104, to or from therapeutic element 114, and/or for other reasons.

In examples, catheter system 108 (e.g., elongate body 110) defines an inlet lumen 162 and an outlet lumen 164 configured to provide at least a portion of the flow path. Inlet lumen 162 and outlet lumen 164 are fluidically coupled to interior volume 116, such that a fluid may flow into interior volume 116 via inlet lumen 162, flow through some portion of interior volume 116, and flow out of interior volume 116 via outlet lumen 164. For example, inlet lumen 162 may be configured to provide a flow path for an inlet flow F1 (FIG. 5) from fluid inlet 118 to interior volume 116. Outlet lumen 164 may be configured to provide a flow path for an outlet flow F2 from interior volume 116 to fluid outlet 120. Inlet flow F1 may be some portion of or substantially all of a pump flow FP provided by pump 124 to fluid inlet 118. Outlet flow F2 may be some portion of or substantially all of a valve flow FV issuing from fluid outlet 120 and flowing through valve 122.

In examples, inlet lumen 162 defines an proximal opening 166 (“inlet proximal opening 166”) and an distal opening 168 (“inlet distal opening 168”) fluidically coupled to inlet proximal opening 166. Inlet distal opening 168 is open to interior volume 116. Outlet lumen 164 may define a distal opening 170 (“outlet distal opening 170”) and a proximal opening 172 (“outlet proximal opening 172”) open to outlet distal opening 170. Outlet distal opening 170 may be fluidically coupled to interior volume 116. In examples, catheter system 108 includes an inlet conduit 174 that fluidically couples inlet proximal opening 166 and an outlet 176 of pump 124 (“pump outlet 176”) and an outlet conduit 176 that fluidically couples outlet proximal opening 172 and an inlet 180 of valve 122 (“valve inlet 180”). Inlet conduit 174 and outlet conduit 176 can be respective lumens of elongate body 110 in some examples or can be separate from elongate body 110 in other examples.

Catheter system 108 may be configured such that a fluid flowing through inlet conduit 174, inlet lumen 162, interior volume 116, outlet lumen 164, and/or outlet conduit 176 is fluidically isolated from blood vessel 102 when catheter system 108 (e.g., distal body portion 110A) is positioned within blood vessel 102. In examples, the flow path defined by catheter system 108 includes at least inlet lumen 162, interior volume 116, and outlet lumen 164. In some examples, the flow path defined by catheter system 108 includes inlet conduit 174 and/or outlet conduit 178.

Catheter system 108 defines flow area A (FIG. 5) to cause the fluid in the flow path to exert the inflation pressure PI within interior volume 116. Catheter system 108 is configured such that flow area A presents a flow restriction to the fluid flow, such that flow area A causes the fluid to generate a differential pressure across the flow area A as the fluid flows through flow area A. Catheter system 108 may be configured such that the differential pressure controls the inflation pressure PI within interior volume 116. Control circuitry 128 is configured to adjust the flow area A and/or a flow rate provided by pump 124 to adjust the differential pressure generated by the fluid flow to, for example, control the inflation pressure PI exerted by the fluid within interior volume 116. Hence, catheter system 108 may be configured to control a size (e.g., a diameter) defined by balloon 112 by at least adjusting the flow area A defined.

In some examples, flow area A is downstream of interior volume 116 when the fluid flows upstream from inlet proximal opening 166, through interior volume 116, and to outlet proximal opening 172. Flow area A is configured such that the fluid generates a pressure PB (e.g., a back pressure) (FIG. 5) within outlet conduit 178 and/or outlet lumen 164 as the fluid flows through flow area A. Catheter system 108 may be configured such that the pressure PB influences the inflation pressure PI. In examples, catheter system 108 is configured such that the fluid flowing downstream of interior volume 116 transfers at least some portion of the pressure PB to interior volume 116. Hence, control circuitry 128 may control the inflation pressure PI (and the corresponding size of balloon 112) by at least controlling the pressure PB generated by the fluid as the fluid flows through the flow area A. In other examples, flow area A is upstream of interior volume 116, within interior volume 116, or within some other location within the flow path defined by catheter system 108.

Control circuitry 128 is configured to adjust the flow area A and/or a flow rate provided by pump 124 to adjust the inflation pressure PI within interior volume 116. For example, catheter system 108 may be configured such that altering the defined flow area A alters the flow restriction on the fluid passing through flow area A, thereby altering the pressure PB. Catheter system 108 may be configured such that altering the flow rate provided by pump 124 alter the mass flow through the defined flow area A, thereby altering the pressure PB. Altering the pressure PB may alter the inflation pressure PI exerted by the fluid within interior volume 116. For example, catheter system 108 can be configured such that a decrease in an area described by flow area A increases the flow restriction on the fluid passing through flow area A, increasing the pressure PB and causing an increase in the inflation pressure PI. In addition, catheter system 108 can be configured such that an increase in an area described by flow area A decreases the flow restriction on the fluid passing through flow area A, decreasing the pressure PB and causing a reduction in the inflation pressure PI. Catheter system 108 can be configured such that an decrease in the flow provided by pump 124 decreases the pressure PB and causes a reduction in the inflation pressure PI, and/or such that an increase in the flow provided by pump 124 increases the pressure PB and causes an increase in the inflation pressure PI. In examples, control circuitry is configured to control one or both of valve 122 and/or pump 124 to adjust the inflation pressure PI. Hence control circuitry 128 may be configured to control the inflation pressure PI within interior volume 116, and hence a size (e.g., a diameter) defined by balloon 106, by at least controlling a size of flow area A defined by catheter system 108.

In examples, valve 122 defines the flow area A. In some examples, valve 122 is fluidically coupled to outlet lumen 164 and/or outlet conduit 178 such that valve flow FV flows into valve inlet 180 and at least partially through flow area A before discharging from an outlet 182 of valve 122 (“valve outlet 182”). In examples, valve outlet 182 configured to direct valve flow FV to a discharge reservoir (not shown). Valve inlet 180 and/or valve outlet 182 may be defined by a body 184 of valve 122 (“valve body 184”). Valve 122 can have any suitable configuration configured to control fluid flow. In examples, valve 122 includes a restricting element 186 (e.g., a valve disc) configured to at least partially define the flow area A. Restricting element 186 may be configured to move relative to a portion of a valve body 184 (e.g., relative to a valve seat) to define the flow area A. In examples, valve 122 is configured such that a size (e.g., an area) of flow area A is a function of a position of restricting element 162 relative to valve body 184. Hence, control circuitry 128 can be configured to control a position of valve 122 (e.g., a position of restricting element 186 relative to valve body 184) to control the flow area A, and thus control the inflation pressure PI within interior volume 116 based on the position of valve 122.

In examples, valve 122 includes an actuator 188 configured to position valve 122 (e.g., configured to position restricting element 162 relative to valve body 184). In examples, actuator 188 is configured to position and/or alter a position of valve 122 based on a valve signal received from control circuitry 128 via signal link 190 (e.g., an electrical, electronic, hydraulic, pneumatic, or other type of signal).

Valve 122 may be any type of valve sufficient to define and/or alter flow area A. Valve 122 may be, for example, a globe valve, poppet valve, a needle valve, a gate valve, a spool valve, or some other mechanism or combination of mechanisms sufficient to define flow area A. In examples, valve 122 may be a remotely actuated valve. Actuator 188 may be any type of actuator sufficient to cause valve 122 (e.g., restricting element 186) to define and/or alter the flow area A. In examples, actuator 188 comprises a solenoid actuator configured to influence the position of valve 122. Actuator 188 may be configured to position and/or alter a position of valve 122 in response to a received electrical or electronic communication (e.g., received via signal link 190). In some examples, actuator 188 is configured to position and/or alter a position of valve 122 based on a supply of a control fluid. For example, valve 122 may be a hydraulically or pneumatically operated valve. Valve 122 may include control circuitry configured to control components of valve 122 in response to a received valve signal. The control circuitry may be some portion of control circuitry 128 or may be substantially separate from control circuitry 128. Valve 122 may be configured to provide communications to other devices in data communication with control circuitry 128.

Catheter system 108 may be configured to enhance the impact of altering the flow area A on the inflation pressure PI exerted within interior volume 116. In examples, catheter system 108 defines the flow path for the fluid such that different sections of the flow path define differing resistance coefficients, where the resistance coefficients describe a resistance to the fluid flow through the respective sections. The resistance coefficient (e.g., a resistance coefficient K) for a given section may be, for example, directly proportional to a length of the given section, directly proportional to a friction factor of the given section, and/or inversely proportional to a diameter and/or equivalent diameter of the given section. Catheter system 108 may define flow area A (e.g., using valve 122) at a particular location within the flow path to enhance the enhance the impact of altering the flow area A on the inflation pressure PI, based on the differing resistance coefficients. For example, catheter system 108 may define the flow path such that valve 122 is located upstream of a section of the flow path having a lower flow coefficient, such that increases and decreases in the flow area A have a greater impact on the inflation pressure PI compared to locating valve 122 upstream of section having a higher flow coefficient.

For example, catheter system 108 may define the flow path such that a first section 189 of the flow path defines a first resistance coefficient K1 and a second section 191 of the flow path defines a second resistance coefficient K2. The second resistance coefficient K2 may be less than the first resistance coefficient K1. Catheter system 108 may be configured such that flow area A is defined (e.g., by valve 122) upstream of second section 191 when the fluid flows in a direction from inlet conduit 174 and/or inlet lumen 162 to outlet lumen 164 and/or outlet conduit 178. In examples, second section 191 is upstream of interior volume 116. In examples, first section 189 is defined by at least some portion of inlet lumen 162 and/or at least some portion of inlet conduit 174. Second section 191 may be defined by at least some portion of outlet lumen 164 and/or at least some portion of outlet conduit 178.

In some examples, catheter system 108 is configured such that pump 124 (e.g., pump outlet 176) is downstream of first section 189. Catheter system 108 may be configured such that a flow from pump 124 (e.g., flow FP and/or flow F1) flows through first section 189 prior to entering interior volume 116. Positioning pump 124 downstream of first section 189 may enhance the impact of altering the flow area A on the inflation pressure PI. For example, when pump 124 is downstream of first section 189 and the fluid within the flow path is an incompressible fluid (e.g., saline, or another incompressible fluid), defining the flow area A upstream of second section 191 may allow alterations in flow area A have a greater impact on the back pressure PB, such that that the alterations in flow area A have a greater impact on the inflation pressure PI.

Catheter system 108 may be configured to cause first section 189 to define the first resistance coefficient K1 and/or cause second section 191 to define the second resistance coefficient K2 in any manner. For example, a first cross-sectional dimension (e.g., a first diameter) of inlet lumen 162 can be less than a second cross-sectional dimension (e.g., a second diameter) of outlet lumen 164. In addition to or instead of the cross-sectional dimensions of lumens 162, 164, in some examples, a first length of first section 189 (e.g., a length of a flow path for flow F1) is greater than a second length of second section 191 (e.g., a length of a flow path for flow F2). In some examples, first section 189 has a first friction factor greater than a second friction factor of second section 191. Catheter system 108 may include one or more flow restricting devices (e.g., orifices, baffles, or other flow restricting devices) configured to cause the first resistance coefficient K1 to exceed the second flow resistance coefficient K2. For example, catheter system 108 may include one or more flow restricting devices within inlet lumen 162 and/or inlet conduit 174. Any of the aforementioned techniques for controlling the resistance coefficients K1, K2 can be used alone or in combination with one or more of the other techniques.

Sensor 126 is configured to sense a parameter indicative of the inflation pressure PI within interior volume 116 and generate a pressure signal indicative of the inflation pressure PI. The pressure indicative of the inflation pressure PI may be a pressure at a location along the flow path defined by catheter system 108. In examples, the pressure indicative of the inflation pressure PI is a pressure of a fluid within and/or fluidically coupled to interior volume 116. For example, sensor 126 may be configured to generate a pressure signal indicative of a pressure of the fluid within interior volume 116, outlet lumen 164, outlet conduit 178, valve 122, inlet lumen 162, inlet conduit 174, and/or some fluid fluidically coupled to interior volume 116. Catheter system 108 can be configured to adjust the flow area A (e.g., using a position of valve 122) to increase, decrease, and/or maintain the inflation pressure PI within balloon 112 based on the pressure signal generated by sensor 126.

Sensor 126 may be configured to generate the pressure signal as a function of a fluid pressure imposed on some portion of sensor 126. Sensor 126 may be configured to use any type of force collector to sense the outlet pressure, including, for example, diaphragms, pistons, bourdon tubes, bellows, or some other collector. Sensor 126 may transduce the pressure into an electrical signal using, for example, piezoresistive strain gauges, capacitors, electromagnets, optical fibers, potentiometric wipers, or other devices. Sensor 126 may be configured to sense a static pressure, a dynamic pressure, and/or a total pressure. Sensor 126 may be configured to sense an absolute pressure or a gauge pressure. The pressure signal generated by sensor 126 may be an analog electrical signal or a digital signal. In some examples, sensor 126 includes processing circuitry configured to interpret a response of the force collector and generate the pressure signal, and/or control circuitry 128 may be configured to interpret a response of the force collector and generate the pressure signal. Sensor 126 may be configured to provide and/or otherwise communicate the pressure signal to other devices of medical system 100 in data communication with sensor 126.

Control circuitry 128 is configured to receive the pressure signal from sensor 126 and cause an adjustment in the inflation pressure PI within interior volume 116 based on the pressure signal. In examples, control circuitry 128 is configured to receive the pressure signal from sensor 126 via a communication link 192. Control circuitry 128 may be configured to receive signal indicative of a flow rate from sensor 129 via a communication link 193. In examples, control circuitry 128 is configured to receive an input signal indicative of the pressure setpoint from user interface 130 (e.g., via communication link 194). Control circuitry 128 may be configured to cause an adjustment in the flow area A to adjust the inflation pressure PI based on a comparison of (e.g., difference between) a pressure indicated by the pressure signal and the pressure setpoint indicated by the input signal. In examples, control circuitry 128 is configured to adjust a position of valve 122 (e.g., restricting element 186) based on the difference between the pressure indicated by the pressure signal and the pressure setpoint indicated by the input signal. Control circuitry 128 may be configured to communication with actuator 188 (e.g., via signal link 190) to cause actuator 188 to position valve 122.

In examples, the input signal is indicative of a specific dimension of balloon 112 (e.g., one of the specific dimensions D1, D2, D3, . . . , DN (FIG. 6)). Control circuitry 128 may be configured to define the pressure setpoint using the input signal indicative of a specific balloon dimension. For example, control circuitry 128 may be configured to define the pressure setpoint as one of a specific pressure (e.g., one of the specific pressures P1, P2, P3, . . . , PN) corresponding to the specific dimension indicated by the input signal. For example, control circuitry 128 may be configured to define the specific pressure using the correlation C1 (FIG. 6). As an example, control circuitry 128 may be configured to define the specific dimension using a table stored in a memory, wherein the table correlates a specific dimension to a specific pressure.

Control circuitry 128 may be configured to cause an adjustment in the flow area A to adjust the inflation pressure PI acting within interior volume 116 based on the pressure setpoint defined. For example, control circuitry may adjust the flow area A (e.g., using valve 122) to increase the inflation pressure PI when sensor 126 indicates the inflation pressure PI is less than the pressure setpoint. Control circuitry 128 may adjust the flow area A (e.g., using valve 122) to decrease the inflation pressure PI when sensor 126 indicates the inflation pressure PI is greater than the pressure setpoint. Control circuitry 128 may be configured to substantially maintain the inflation pressure PI (e.g., maintain or nearly maintain to the extent permitted by manufacturing tolerances) when the inflation pressure PI is substantially equal to the pressure setpoint or within a range of values defined around the pressure setpoint. For example, control circuitry 128 may be configured to maintain the inflation pressure PI within 1% to about 30% of the pressure setpoint, such as within 30% of the pressure setpoint, within 20% of the pressure setpoint, within 10% of the pressure setpoint, within 5% of the pressure setpoint, or within 1% of the pressure setpoint. In examples, control circuitry 128 positions valve 122 to adjust the pressure PB (FIG. 5) caused by the position of valve 122 in order to increase, decrease, and/or substantially maintain the inflation pressure PI.

As noted above, in some examples, system 100 includes user interface 130, which is configured to receive the input indicative of the pressure setpoint (e.g., from a user) and provide an input signal to control circuitry 128 (e.g., via communication link 196) based on the indicative input. The indicative input may be, for a dimension of balloon 112 (e.g., a diameter or other dimension), a pressure value, or some other input indicative of the pressure setpoint. User interface 130 may be configured to receive the indicative input via a button or keypad, a touch screen, a speaker configured to receive voice commands from a user, and/or a display. In some examples, user interface 130 may include or be configured to be used in combination with a mobile phone, smartphone, tablet computer, personal computer, desktop computer, personal digital assistant, router, modem, remote server or cloud computing device, and/or related device. In some examples, positioning member 134 is configured to move (e.g., rotate) relative to device body 132 to cause user interface 130 to provide the indicative input (e.g., a desired size of balloon 112, a pressure value, and/or some other input indicative of the pressure setpoint). In some examples, user interface 130 is configured to display information, such as one or more desired sizes and/or setpoints (e.g., the current desired size and/or setpoint being used by control circuitry 128 or one or more predetermined desired sizes and/or setpoints from which the user can select to input a desired size of balloon 112).

Communication link 192, 193, 194, 196 and/or signal link 190 may be hard-line and/or wireless communications links. In some examples, communication link 192, 193, 194, 196 and/or signal link 190 may comprise some portion of control circuitry 128. In some examples, communication link 192, 193, 194, 196 and/or signal link 190 comprise a wired connection, a wireless Internet connection, a direct wireless connection such as wireless LAN, Bluetooth™, Wi-Fi™, and/or an infrared connection. Communication link 192, 193, 194, 196 and/or signal link 190.

In some examples, control circuitry 128 is configured to control an inflation of balloon 112 based on an assessment that balloon 112 has expanded sufficiently to contact vessel wall 104. For example, balloon 112 may be configured such that, as balloon 112 expands in a substantially unconstrained state (e.g., prior to contacting vessel wall 104), inflation pressure PI within interior volume 116 defines a pressure trend profile, such as the pressure trend profile described by correlation C1 (FIG. 6). Control circuitry 128 may be configured to determine and/or monitor a trend of the inflation pressure PI (“inflation pressure trend”) to determine if balloon 112 may have contacted vessel wall 104, and/or if an expansion of balloon 112 is otherwise constrained. In examples, control circuitry 128 is configured to receive and/or request a plurality of pressure signals from sensor 126 over a time period and determine the inflation pressure trend using at least the plurality of pressure signals.

In examples, instead of or in addition to receiving an input signal indicative of a pressure set point from user interface 130, control circuitry 128 is configured to establish the pressure setpoint for the inflation pressure PI based on the assessment that balloon 112 has expanded sufficiently to contact vessel wall 104. Control circuitry 128 may be configured to determine a resulting inflation pressure (e.g., using a pressure signal from sensor 126) when control circuitry 128 assesses balloon 112 has contacted vessel wall 104, and base the pressure setpoint on the resulting inflation pressure. Thus, in some examples, control circuitry 128 is configured to establish the pressure setpoint for the inflation pressure PI without need for an input signal from user interface 130, potentially easing the performance of a medical procedure within blood vessel 102.

Control circuitry 128 may be configured to identify a departure (e.g., a relatively sudden increase) from an inflation pressure trend to assess balloon 112 may have expanded sufficiently to contact vessel wall 104 (e.g., to assess that vessel wall 104 may be constraining the continued expansion of balloon 112). In some examples, control circuitry 128 assesses that balloon 112 is constrained by vessel wall 104 based on a departure between the inflation pressure trend and a pressure trend profile described by correlation C1. Control circuitry 128 may be configured to alter the flow area A (e.g., a position of valve 122) in response to the departure from the inflation pressure trend and/or the pressure trend profile of correlation C1. In examples, control circuitry 128 is configured to identify the inflation pressure PI when the departure occurs and establish a pressure setpoint based on the identified inflation pressure, such that, for example, catheter system 108 substantially maintains an inflation pressure PI within interior volume 116 sufficient to keep balloon 112 in contact with vessel wall 104.

For example, control circuitry 128 may be configured to receive and/or request a first plurality of pressure signals from sensor 126 indicating inflation pressures such as S1, S2, S3, and S4 (FIG. 6) occurring substantially over a time period T. Control circuitry 128 may determine an inflation pressure trend PT using two or more of the inflation pressures S1, S2, S3, and S4 and the time period T1. Control circuitry 128 may receive and/or request one or more additional pressure signals from sensor 126 subsequent to receiving S1, S2, S3, S4 to assess if a departure from inflation pressure trend PT has occurred. Control circuitry 128 may be configured to extrapolate the inflation pressure trend PT (e.g., extrapolate chronologically) and compare the additional pressure signals to the extrapolated inflation pressure trend to assess whether balloon 112 may have expanded sufficiently to contact vessel wall 104.

For example, control circuitry 128 can be configured to receive and/or request additional pressure signals indicating inflation pressures such as S5 and/or S6 and compare inflation pressures S5, S6 to inflation pressure trend PT and determine that inflation pressure S5, S6 substantially match (e.g., match within a defined range) pressures expected from an extrapolation of pressure trend line PT. Control circuitry 128 determines that balloon 112 is continuing to expand in an unconstrained manner (e.g., not in contact with vessel wall 104) based on the inflation pressure S5, S6 substantially matching the extrapolated pressure trend of pressure trend line PT.

Alternately, control circuitry 128 may receive and/or request additional pressure signals indicating inflation pressures such as S7 and/or S8 and compare inflation pressures S7, S8 to inflation pressure trend PT, and determine that inflation pressure S7 and/or S8 depart (e.g., fall outside of the defined range) from the extrapolated pressure trend of pressure trend line PT1. Control circuitry 128 can be configured to determine that the expansion of balloon 112 may be constrained (e.g., in contact with vessel wall 104) based on the inflation pressure S7 and/or S8 departing from the extrapolated pressure trend of pressure trend line PT.

In some examples, control circuitry 128 defines the inflation pressure trend PT using correlation C1. For example, control circuitry 128 may define the inflation pressure trend PT1 over an interval defined by two or more of S1, S2, S3, S4, S5, S6, S7, and S8. Control circuitry 128 may be configured to determine that balloon 112 is continuing to expand in an unconstrained manner (e.g., not in contact with vessel wall 104) based on inflation pressure S1, S2, S3, S4, S5, S6, S7, and/or S8 substantially matching (e.g., matching within a defined range) pressures expected by correlation C1. Control circuitry 128 may be configured to determine that the expansion of balloon 112 may be constrained (e.g., in contact with vessel wall 104) based on inflation pressure S1, S2, S3, S4, S5, S6, S7, and/or S8 departing (e.g., falling outside of the defined range) from pressures expected by correlation C1.

In examples, control circuitry 128 is configured to define a pressure trend PT determined from correlation C1 based on a flow rate of the fluid flowing through inlet conduit 174, inlet lumen 162, interior volume 116, outlet lumen 164, and/or outlet conduit 178. Control circuitry 128 may be configured to determine a flow rate of the fluid using, for example, a flow detector within catheter system 108, a speed of pump 124, a position of valve 122, and/or other parameters. Control circuitry 128 may be configured to map the correlation C1 to the flow rate and/or a time axis defined by the flow rate in order to, for example, correlate an inflation pressure trend PT defined by correlation C1 with the flow rate through inlet conduit 174, inlet lumen 162, interior volume 116, outlet lumen 164, and/or outlet conduit 178.

Hence, catheter system 108 may be configured to control a size (e.g., a dimension) of balloon 112 by at least controlling the inflation pressure PI acting within interior volume 116. In this way, catheter system 108 may adjust the size of balloon 112 to account for the size of a blood vessel 102 in which elongate body 110 and balloon 112 are positioned. Catheter system 108 may be receive an input (e.g., from a clinician via user interface 130) indicative of the size (e.g., a diameter) of a blood vessel in which a procedure is to be performed. Once positioned within the blood vessel, control circuitry 128 may be configured to inflate balloon 112 to a particular inflation pressure PI based on the input, such that the dimension of balloon 112 enables balloon 112 to be in apposition with the blood vessel walls during the procedure. In examples, control circuitry 128 is configured to at least partially deflate balloon 112 (e.g., based on received user input) to enable a clinician to reposition catheter system 108 within patient 106.

In addition to or instead of controlling the inflation pressure PI to control the size of balloon 112, in some examples, control circuitry 128 is configured to control an energy level of therapeutic element 114 based on the size of balloon 112. Catheter system 108 may be configured such that the displacement B2 (FIGS. 2 and 5) between therapeutic element 114 and vessel wall 104 is proportional to and/or substantially dependent on the size of balloon 112 within blood vessel 102. For example, catheter system 108 may be configured to support therapeutic element 114 within interior volume 116, or at some other location wherein the displacement B2 is proportional to and/or substantially dependent on the size of balloon 112.

In examples, therapeutic element 114 is configured to transmit energy (e.g., acoustic energy, microwave energy, RF energy, or another energy form) to deliver a treatment to vessel wall 103. The energy transmitted may be a function of the power level of therapeutic element 114. In examples, therapeutic element 114 may be configured such that power level necessary for delivery of the treatment is dependent on the displacement B2 defined by balloon 112. Thus, in some examples, control circuitry 128 is configured to determine a size (e.g., a diameter) and/or inflation pressure of inflated balloon 112 and determine a power level for therapeutic element 114 based on the size and/or inflation pressure. In some examples, control circuitry 128 is further configured to determine the power level based on a type of media (e.g., saline inflating balloon 112) through which therapeutic element 114 transmits energy over the displacement B2.

In examples, control circuitry 128 is configured to control the energy level of therapeutic element 114 using the input signal (e.g., from user interface 130) indicative of a specific dimension of balloon 112 (e.g., one of the specific dimensions D1, D2, D3, . . . , DN). For example, control circuitry 128 can be configured to control the energy level of therapeutic element 114 based on the specific dimension and/or a parameter determined using the specific dimension (e.g., a volume of interior volume 116). In some examples, control circuitry 128 is configured to control the energy level of therapeutic element 114 based on the pressure signal from sensor 126 (e.g., when the pressure signal is indicative of a size of balloon 112). For example, control circuitry 128 may be configured such that indications of a larger size of balloon 112 causes control circuitry 128 to control the energy level of therapeutic element 114 to a relatively higher power level. Control circuitry 128 may be configured such that indications of a smaller size of balloon 112 causes control circuitry 128 to control the energy level of therapeutic element 114 to a relatively lower power level (e.g., a power level less than the higher power level).

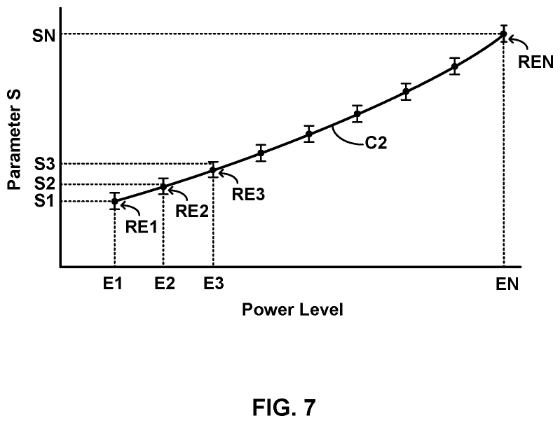

As an example, FIG. 7 illustrates an example relationship between a parameter S indicative of a size of balloon 112 and a power level of therapeutic element 114. In some examples, control circuitry 128 is configured to define the parameter S based on, for example, a specific dimension of balloon 112, a parameter determined using the specific dimension (e.g., a volume), a pressure signal from sensor 126, an input signal from user interface 130, and/or other indications. The specific parameter S may be proportion to a size (e.g., a specific dimension) of balloon 112. In examples, the specific parameter S increases when the size of balloon 112 increases. Control circuitry 128 may be configured to control the power level of therapeutic element 114 based on the parameter S.

Control circuitry 128 may be configured to define one or more specific power levels such as E1, E2, E3, . . . , EN when the parameter S describes a specific parameter such as S1, S2, S3, . . . , SN, respectively. For example, control circuitry 128 may be configured such that specific parameter S1 causes control circuitry 128 to control the power level of therapeutic element 114 at about the specific power level E1, specific parameter S2 causes control circuitry 128 to control the power level of therapeutic element 114 at about the specific power level E2, specific parameter S3 causes control circuitry 128 to control the power level of therapeutic element 114 at about the specific power level E3, specific parameter SN causes control circuitry 128 to control the power level of therapeutic element 114 at about the specific power level EN, and so on. In examples, specific power level E1 is a power level within a range RE1, specific power level E2 is a power level within a range RE2, specific power level E3 is a power level within a range RE3, and/or specific power level E4 is a dimension with a range REN.

In examples, the specific power levels E1, E2, E3, . . . , EN increase as the specific parameters S1, S2, S3, . . . , SN increase. In some examples, the specific power levels E1, E2, E3, . . . , EN are described by a correlation C2 relating a specific parameter S to a specific power level. In some examples, the correlation C2 is continuous, such that correlation C2 describes a specific power level for any specific parameter S between and including specific parameter S1 and specific parameter SN. The correlation C2 may be a discrete, such that such that correlation C2 describes a specific power level when the specific parameter S defines or is assigned (e.g., by control circuitry 128) a respective discrete value (e.g., one or S1, S2, S3, . . . , SN). In some examples, the correlation C2 may be defined by a data structure, such as a coordinate table, describing a plurality of ordered pairs describing a set of coordinates (e.g., a coordinate describing a specific parameter S and a corresponding coordinate describing a specific power level).

In examples, the correlation C2 may be defined by a plurality of separate correlations defined over one or more intervals, such as an interval between E1 and E2, an interval between E2 and E3, and/or another interval. The correlation C2 may define any curvature and/or slope over any interval, and may be a linear function over one or more intervals. Control circuitry 128 may be configured to control the power level such that therapeutic element 114 substantially achieves a specific power level (e.g., one of E1, E2, E3, . . . , EN) when balloon 112 defines the specific parameter S.

In some examples, control circuitry 128 is configured to determine the power level for therapy delivery by therapeutic element 114 using the input signal from user interface 130 and/or the pressure signal from sensor 126. For example, control circuitry 128 may be configured to determine a specific parameter S (e.g., one of S1, S2, S3, . . . , SN) using the input signal from user interface 130, the pressure signal from sensor 126, and/or the correlation C1 (FIG. 6) and determine a specific power level (e.g., one of the specific power levels E1, E2, E3, . . . , EN) corresponding to the specific parameter S. For example, control circuitry 128 may be configured to determine the specific power level using the correlation C2 (FIG. 7). Control circuitry 128 may be configured to determine the specific power level using a table (or other data structure) stored in a memory, wherein the table correlates a specific parameter S to a specific power level.

In these examples, control circuitry 128 is configured to adjust the power level of therapeutic element 114 based on the specific power level defined. For example, control circuitry 128 may increase the power level when the input signal from user interface 130 and/or the pressure signal from sensor 126 indicates the power level is less than the specific power level (or range of levels) associated with the parameter S defined by balloon 112. Control circuitry 128 may be configured to decrease the power level when the input signal from user interface 130 and/or the pressure signal from sensor 126 indicates the power level is greater than the specific power level associated with the parameter S defined by balloon 112. Control circuitry 128 may be configured to substantially maintain the power level (e.g., maintain or nearly maintain to the extent permitted by manufacturing tolerances) when the power level is substantially equal to the specific power level associated with the parameter S defined by balloon 112. For example, control circuitry 128 may be configured to maintain the power level of therapeutic element 114 within 1% to about 30% of a specific power level, such as within 30% of the specific power level, within 20% of the specific power level, within 10% of the specific power level, within 5% of the specific power level, or within 1% of the specific power level.

In examples, control circuitry 128 is configured to communicate control therapeutic element 114 (e.g., via communication link 194) to cause therapeutic element 114 to alter and/or substantially maintain a power level. In examples, catheter system 108 (e.g., therapeutic element 114 or generator 144) includes element circuitry 198 (FIGS. 4 and 5) configured to cause therapeutic element 114 to emit energy at a specific energy level (e.g., up to about 50 watts, up to about 100 watts, or another power level). Control circuitry 128 may be configured to communicate a signal indicative of a specific power level to element circuitry 198 (e.g., via communication link 194). Element circuitry 198 is configured to cause therapeutic element 114 to emit an amount of power based on the signal indicative of the specific power level. In some examples, control circuitry 128 is configured to communicate with generator 144 (FIG. 1) to cause therapeutic element 114 to alter and/or substantially maintain a power level provided to therapeutic element 114. The power level may define a level for any type of energy, including pulsed field, electrical current, microwave, radiofrequency, monopolar, bipolar energy, and/or others. In examples, control circuitry 128 is configured to communicate with automated control algorithm 146 and/or evaluation/feedback algorithm 148 to alter and/or substantially maintain a power level.

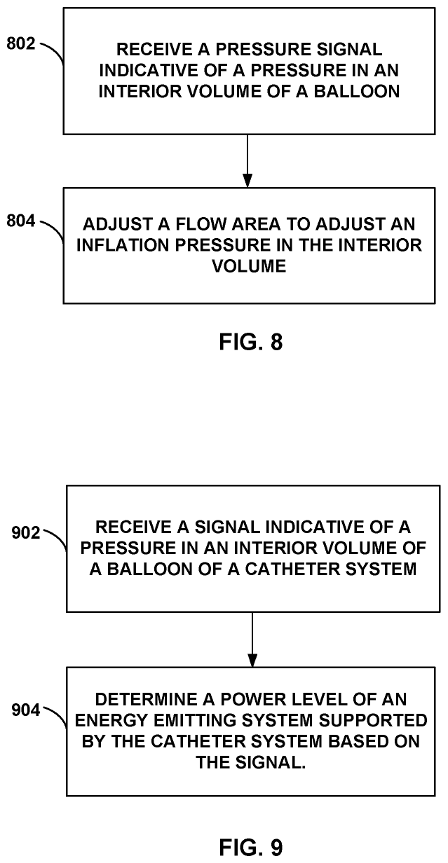

An example technique for controlling the size of a balloon 112 is illustrated in FIG. 8. Although the technique is described mainly with reference to medical system 100 of FIGS. 1-5, the technique may be applied to other medical systems in other examples and by control circuitry 128 alone or in combination with other control circuitry in other examples.

The technique includes receiving, by control circuitry 128 of a catheter system 108, a pressure signal from a sensor 126 indicative of a pressure of a fluid flowing through an interior volume 116 of balloon 112 (802). In some examples, the pressure is indicative of a pressure of the fluid within interior volume 116 or elsewhere along a flow path for fluid through interior volume 116. Balloon 112 may be positioned within a blood vessel 102 of a patient 106 using an elongate body 110 (e.g., distal body portion 110A) supporting balloon 112. The fluid flowing through interior volume 116 may exert an inflation pressure PI within interior volume 116 (e.g., exert the inflation pressure PI on balloon interior surface 140).

The technique includes adjusting a flow area A for the fluid flow, using control circuitry 128, to adjust the inflation pressure PI within interior volume 116 (804). Control circuitry 128 may control the inflation pressure PI to control the size of balloon 112. For example, control circuitry 128 may position valve 122 to control the flow area A. In examples, valve 122 defines the flow area A downstream of interior volume 116 when the fluid flows through interior volume 116.

Sensor 126 may sense a parameter indicative of the inflation pressure PI of balloon 112 and generate a pressure signal indicative of the inflation pressure PI. Control circuitry 128 may adjust a position of valve 122 to increase, decrease, and/or maintain the inflation pressure PI within balloon 112 based on the pressure signal generated by sensor 126. In examples, control circuitry 128 receives an input indicative of a pressure setpoint from a user interface 130. Control circuitry 128 may control the flow area A based on a comparison of the pressure signal and the pressure setpoint. In some examples, the input indicative of the pressure setpoint describes a size (e.g., a diameter) of balloon 112. In some examples, the input indicative of the pressure setpoint describes a pressure (e.g., an inflation pressure PI) for balloon 112. User interface 130 may receive the input indicative of the pressure setpoint from a user and provide the input to control circuitry 128.

Control circuitry 128 may receive the pressure signal from sensor 126 and cause an adjustment in the inflation pressure PI within interior volume 116 based on the pressure signal. Control circuitry 128 may cause an adjustment in the flow area A to adjust the inflation pressure PI based on a comparison of a pressure indicated by the pressure signal and the pressure setpoint indicated by the input signal. In examples, control circuitry 128 adjusts a position of valve 122 (e.g., restricting element 186) based on the difference between the pressure indicated by the pressure signal and the pressure setpoint indicated by the input signal. In examples, control circuitry 128 communicates with an actuator 188 to cause actuator 188 to position valve 122.

In examples, control circuitry 128 may define the pressure setpoint as a specific pressure (e.g., one of the specific pressures P1, P2, P3, . . . , PN) corresponding to a specific dimension. Control circuitry 128 define the specific pressure using a correlation C1, or a table stored in a memory, of using some other method. Control circuitry 128 may cause an adjustment in the flow area A to adjust the inflation pressure PI acting within interior volume 116 based on the pressure setpoint defined. For example, control circuitry may adjust the flow area A (e.g., using valve 122) to increase the inflation pressure PI when sensor 126 indicates the inflation pressure PI is less than the pressure setpoint. Control circuitry may adjust the flow area A (e.g., using valve 122) to decrease the inflation pressure PI when sensor 126 indicates the inflation pressure PI is greater than the pressure setpoint. Control circuitry 128 may be configured to substantially maintain the inflation pressure PI (e.g., maintain or nearly maintain to the extent permitted by manufacturing tolerances) when the inflation pressure PI is substantially equal to the pressure setpoint or within a range of values defined around the pressure setpoint.

In examples, instead of or in addition to receiving an input signal indicative of a pressure set point from user interface 130, control circuitry 128 establishes the pressure setpoint for the inflation pressure PI. Control circuitry 128 may establish the pressure setpoint based on the assessment that balloon 112 has expanded sufficiently to contact vessel wall 104. Control circuitry 128 may determine a resulting inflation pressure (e.g., using a pressure signal from sensor 126) when control circuitry 128 assesses balloon 112 has contacted vessel wall 104, and base the pressure setpoint on the resulting inflation pressure.

Control circuitry 128 may identify a departure (e.g., a relatively sudden increase) from an inflation pressure trend to assess balloon 112 may have expanded sufficiently to contact vessel wall 104. In examples, control circuitry 128 assesses that balloon 112 is constrained by vessel wall 104 based on a departure between the inflation pressure trend and a pressure trend profile described by correlation C1. Control circuitry 128 may alter the flow area A (e.g., a position of valve 122) in response to the departure from the inflation pressure trend and/or the pressure trend profile of correlation C1. Control circuitry 128 may identify the inflation pressure PI when the departure occurs and establish a pressure setpoint based on the identified inflation pressure, such that, for example, catheter system 108 substantially maintains an inflation pressure PI within interior volume 116 sufficient to keep balloon 112 in contact with vessel wall 104. In examples, control circuitry 128 determines an inflation pressure trend PT using any of the techniques described above.

An example technique for controlling a power level of therapeutic element 114 is illustrated in FIG. 9. Although the technique is described mainly with reference to medical system 100 of FIGS. 1-5, the technique may be applied to other medical systems in other examples and by control circuitry 128 alone or in combination with other control circuitry in other examples.

The technique includes receiving, by control circuitry 128, a pressure signal indicative of a pressure in interior volume 116 of balloon 112 (902). Control circuitry 128 may receive the pressure signal from sensor 126. Control circuitry 128 may determine a size (e.g., a diameter) of balloon 112 using the pressure signal and control an energy level of therapeutic element 114 based on the size of balloon 112. As noted above, in some examples in which therapeutic element 114 is positioned within interior volume 116, a displacement B2 is proportional to and/or substantially dependent on the size of balloon 112. The power level necessary for delivery of the treatment by therapeutic element 114 can be dependent on the displacement B2 defined by balloon 112. In some examples, the power level necessary for delivery of the treatment is also dependent on a type of media (e.g., saline inflating balloon 112) through which therapeutic element 114 transmits energy over the displacement B2.

In examples, control circuitry 128 determines the energy level of therapeutic element 114 using the input signal (e.g., from user interface 130) indicative of a specific dimension of balloon 112 (e.g., one of the specific dimensions D1, D2, D3, . . . , DN). Control circuitry 128 may control the energy level of therapeutic element 114 based on the specific dimension and/or a parameter determined using the specific dimension (e.g., a volume of interior volume 116). In some examples, control circuitry 128 controls the energy level of therapeutic element 114 based on the pressure signal from sensor 126. Control circuitry 128 may control the energy level of therapeutic element 114 to a relatively higher power level for a larger size of balloon 112 and to a relatively lower power level for a smaller size of balloon 112.

In examples, control circuitry 128 determines a specific parameter S (e.g., one of S1, S2, S3, . . . , SN) using the input signal from user interface 130, the pressure signal from sensor 126, and/or the correlation C1 (FIG. 6). Control circuitry 128 may determine a specific power level (e.g., one of the specific power levels E1, E2, E3, . . . , EN) corresponding to the specific parameter S. In examples, control circuitry 128 determines the specific power level using the correlation C2 (FIG. 7) and/or based on a table or other data structure stored in a memory, wherein the table or other data structure correlates a specific parameter S to a specific power level.

In some examples, control circuitry 128 adjusts the power level of therapeutic element 114 based on the specific power level defined. For example, control circuitry 128 may increase the power level when the input signal from user interface 130 and/or the pressure signal from sensor 126 indicates the power level is less than the specific power level associated with the parameter S defined by balloon 112. Control circuitry 128 may decrease the power level when the input signal from user interface 130 and/or the pressure signal from sensor 126 indicates the power level is greater than the specific power level associated with the parameter S defined by balloon 112. Control circuitry 128 may substantially maintain the power level when the power level is substantially equal to the specific power level associated with the parameter S defined by balloon 112.

Control circuitry 128 may communicate with therapeutic element 114 to cause therapeutic element 114 to alter and/or substantially maintain a power level. In examples, control circuitry 128 communicates element circuitry 198 to cause therapeutic element 114 to emit energy at a specific energy level. In some examples, control circuitry 128 communicates with generator 144 (FIG. 1) to cause therapeutic element 114 to alter and/or substantially maintain a power level provided to therapeutic element 114. The power level may define a level for any type of energy, including pulsed field, electrical current, microwave, radiofrequency, monopolar, bipolar energy, and/or others. In examples, control circuitry 128 communicates with automated control algorithm 146 and/or evaluation/feedback algorithm 148 to alter and/or substantially maintain a power level.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), ferroelectric RAM, a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

In some examples, the disclosure may be summarized in the following numbered clauses.

Clause 1: A catheter system comprising: an elongate body configured to be positioned within a blood vessel of a patient; a balloon defining an interior volume; a sensor configured to generate a pressure signal indicative of a pressure of a fluid within a flow path, the flow path including the interior volume and at least one lumen defined by the elongate body; a valve configured to define a flow area for the fluid; and control circuitry configured to: determine a pressure setpoint, receive the pressure signal from the sensor, and position the valve to adjust the flow area based on the pressure setpoint and the pressure indicated by the sensor signal.

Clause 2: The catheter system of clause 1, wherein the at least one lumen comprises an inlet lumen and an outlet lumen, and wherein the flow path is defined through the inlet lumen and the outlet lumen.

Clause 3: The catheter system of clause 2, wherein a diameter of the outlet lumen is greater than a diameter of the inlet lumen.

Clause 4: The catheter system of clause 2 or clause 3, wherein the pressure signal is indicative of the pressure of the fluid within the outlet lumen.

Clause 5: The catheter system of any one of clauses 2-4, wherein the valve includes a restricting element configured to define the flow area.

Clause 6: The catheter system of any one of clauses 2-5, wherein a first section of the flow path including the inlet lumen defines a first resistance coefficient for the flow, and wherein a second section of the flow path including the outlet lumen defines a second resistance coefficient, wherein the second resistance coefficient is less than the first resistance coefficient.

Clause 7: The catheter system of any one of clauses 1-6, wherein the elongate body supports the balloon.

Clause 8: The catheter system of any one of clauses 1-7, wherein the catheter is configured to receive an input indicative of the pressure setpoint and determine the pressure setpoint using the input.

Clause 9: The catheter system of any one of clauses 1-8, wherein the catheter system is configured to provide a continuous flow through the flow path.

Clause 10: The catheter system of any one of clauses 1-9, wherein the valve includes: a restricting element configured to define the flow area; and a valve actuator configured to position the restricting element, wherein the control circuitry is configured to position the valve to adjust the flow area by at least causing the valve actuator to position the restricting element.

Clause 11: The catheter system of clause 10, wherein the pressure signal is indicative of pressure of the fluid upstream of the restricting element.

Clause 12: The catheter system of any one of clauses 1-11, further comprising a pump configured to cause the fluid to flow through the flow path.

Clause 13: The catheter system of clause 12, wherein the control circuitry is configured to adjust, using the pump, a flow rate of the fluid through the flow path based on the pressure setpoint and the pressure indicated by the sensor signal.

Clause 14: The catheter system of clause 12 or clause 13, wherein the pump defines a pump outlet configured to discharge the fluid into an inlet lumen defined by the elongate body to cause the fluid to flow through the flow path.

Clause 15: The catheter system of any one of clauses 12-14, further comprising a fluid container defining a reservoir configured to hold a volume of the fluid, wherein the pump is configured to draw the fluid from the reservoir and discharge the fluid into the flow path.

Clause 16: The catheter system of any one of clauses 12-15, wherein the pump includes a centrifugal pump.

Clause 17: The catheter system of any one of clauses 12-15, wherein the pump includes a positive displacement pump.

Clause 18: The catheter system of any one of clauses 1-17, wherein the elongate body defines a longitudinal axis, the catheter system further comprising a therapeutic element mechanically supported by the catheter system, wherein the therapeutic element is configured to emit energy in a direction away from the longitudinal axis.

Clause 19: The catheter system of clause 18, wherein the therapeutic element is configured to emit ultrasound energy.

Clause 20: The catheter system of clause 18 or clause 19, wherein a distal portion of the elongate body supports the therapeutic element.

Clause 21: The catheter system of any one of clauses 18-20, wherein the therapeutic element is positioned within the interior volume of the balloon.

Clause 22: The catheter system of any of clauses 18-21, wherein the control circuitry is configured to: determine a power level, and cause the therapeutic element to emit the energy based on the determined power level.

Clause 23: The catheter system of clause 22, wherein the control circuitry is configured to determine the power level based on at least one of the pressure setpoint or the pressure signal indicative of the pressure of the fluid within the flow path.

Clause 24: The catheter system of clause 22 or clause 23, wherein the pressure signal is indicative of the pressure of the fluid within the interior volume, and wherein the control circuitry is configured to determine the power level based on the pressure of the fluid within the internal volume.

Clause 25: The catheter system of any one of clauses 1-24, wherein the balloon is a compliant balloon.

Clause 26: The catheter system of any one of clauses 1-25, wherein the balloon is configured to define a dimension of the balloon based on a pressure of the fluid within the interior volume.

Clause 27: The catheter system of clause 26, wherein the control circuitry is configured to determine the pressure setpoint based on input from a user, wherein the input is indicative of the dimension of the balloon.

Clause 28: The catheter system of clause 26 or 27, wherein the dimension of the balloon is a cross-sectional dimension of the balloon perpendicular to the longitudinal axis.

Clause 29: The catheter system of any one of clauses 1-28, further comprising a user interface configured to receive input indicative of the pressure setpoint from a user and provide the input indicative of the pressure setpoint to the control circuitry.

Clause 30: The catheter system of any one of clauses 1-29, wherein the control circuitry is configured to: determine a pressure trend based on the pressure signal received over a time period, and position the valve based on the pressure trend.

Clause 31: The catheter system of clause 30, wherein the control circuitry is configured to position the valve to adjust the flow area in response to determining a departure of the pressure trend from a pressure trend profile.

Clause 32: A catheter system comprising: an elongate body; a balloon defining an interior volume; a therapeutic element mechanically supported by the elongate body, wherein the therapeutic element is configured to emit energy; and control circuitry configured to: receive an input indicative of a pressure within the interior volume of the balloon, determine a power level based on the indicated pressure, and cause the therapeutic element to emit the energy based on the determined power level.

Clause 33: The catheter system of clause 32, wherein the input includes a pressure setpoint, the catheter system further comprising a user interface configured to receive the pressure setpoint from a user, wherein the control circuitry is configured to determine the power level based on the pressure set point.

Clause 34: The catheter system of clause 32, further comprising a sensor configured to generate a pressure signal indicative of a pressure of a fluid within a flow path defined at least in part by the elongate body, wherein the pressure of the fluid within the flow path is indicative of the pressure within the interior volume of the balloon, and wherein the control circuitry is configured to: receive the pressure signal from the sensor, and determine the power level based on the pressure signal.

Clause 35: The catheter system of clause 34, wherein the control circuitry is configured to: determine a pressure trend based on the pressure signal received over a time period, and adjust a flow area for the fluid based on the pressure trend.

Clause 36: The catheter system of clause 35, wherein the control circuitry is configured to adjust the flow area based on a departure of the pressure trend from a pressure trend profile.

Clause 37: The catheter system of any one of clauses 32-36, wherein the therapeutic element is configured to emit ultrasound energy.

Clause 38: The catheter system of any one of clauses 32-37, further comprising a generator configured to provide energy to the therapeutic element.

Clause 39: The catheter system of clause 38, wherein the control circuitry is configured to control the generator to control the provided energy to cause the therapeutic element to emit the energy at based on the power level.

Clause 40: The catheter system of any one of clauses 32-39, wherein the therapeutic element includes energy control circuitry configured to cause the therapeutic element to emit the energy, and where the control circuitry is configured to control the energy control circuitry to cause the therapeutic element to emit the energy at the power level.

Clause 41: The catheter system of any one of clauses 32-40, further comprising a control device configured to receive a user input and provide the user input to the control circuitry, wherein the control circuitry is configured to alter the energy emitted by the therapeutic element based on the user input.

Clause 42: The catheter system of any one of clauses 32-41, wherein the balloon is a compliant balloon.

Clause 43: The catheter system of any one of clauses 32-42, wherein a dimension of the balloon varies based on the pressure within the interior volume of the balloon, and wherein the input indicative of the pressure within the interior volume of the balloon is indicative of the dimension of the balloon.

Clause 44: The catheter system of clause 43, wherein the elongate body defines a longitudinal axis, and wherein the dimension of the balloon is a cross-sectional dimension of the balloon perpendicular to the longitudinal axis.

Clause 45: The catheter system of any one of clauses 32-44, wherein a distal portion of the elongate body mechanically supports the balloon.

Clause 46: The catheter system of any one of clauses 32-45, wherein a distal portion of the elongate body mechanically supports the therapeutic element.

Clause 47: The catheter system of any one of clauses 32-46, wherein the therapeutic element is positioned within the interior volume of the balloon.

Clause 48: The catheter system of any one of clauses 32-47, further comprising a valve configured to control a pressure of the fluid within a flow path including the interior volume and at least one lumen defined by the elongate body, wherein the control circuitry is configured to position the valve based on the input indicative of the pressure within the interior volume of the balloon.

Clause 49: The catheter system of clause 48, wherein the pressure of the fluid within the flow path is indicative of a pressure upstream of the restricting element.

Clause 50: The catheter system of clause 48 or clause 49, wherein the valve includes: a restricting element configured to define a flow area for the fluid; and a valve actuator configured to position the restricting element, wherein the control circuitry is configured to position the valve by at least causing the valve actuator to position the restricting element.

Clause 51: The catheter system of any one of clauses 47-50, wherein the at least one lumen comprises an inlet lumen and defines an outlet lumen, and wherein the flow path is defined through the inlet lumen and the outlet lumen.

Clause 52: The catheter system of clause 51, wherein a diameter of the outlet lumen is greater than a diameter of the inlet lumen.

Clause 53: The catheter system of clause 51 or clause 52, wherein the sensor of any one of clauses 33-51 is configured to determine the pressure of the fluid within the outlet lumen, and wherein the sensor signal is indicative of the pressure of the fluid within the outlet lumen.

Clause 54: The catheter system of any one of clauses 51-53, wherein a first section of the flow path including the inlet lumen defines a first resistance coefficient for the flow and a second section of the flow path including the outlet lumen defines a second resistance coefficient for the flow, wherein the second resistance coefficient is less than the first resistance coefficient.

Clause 55: The catheter system of any one of clauses 1-54, further comprising a pump configured to cause the fluid to flow through the flow path.

Clause 56: The catheter system of clause 55, wherein the pump defines a pump outlet configured to discharge the fluid into the inlet lumen of any of claims 50-54 to cause the fluid to flow through the flow path.

Clause 57: The catheter system of clause 55 or clause 56, further comprising a fluid container defining a reservoir configured to hold a volume of the fluid, wherein the pump is configured to draw the fluid from the reservoir and discharge the fluid into the flow path.

Clause 58: The catheter system of any one of clauses 55-57, wherein the control circuitry is configured to adjust, using the pump, the flow rate of the fluid through the flow path based on the input indicative of the pressure within the interior volume of the balloon.

Clause 59: A method, comprising: receiving, by control circuitry, a sensor input from a pressure sensor indicative of a pressure of a fluid within a flow path for a fluid flowing through an interior volume of a balloon, the flow path being defined by an elongated body of a catheter, wherein the elongated structure is configured to be positioned with an anatomical lumen of a patient; and positioning, using the control circuitry, a valve configured to throttle a flow of the fluid through the flow path based on a difference between a pressure setpoint and the pressure indicated by the sensor input.

Clause 60: The method of clause 59, wherein the flow path includes an inlet lumen and an outlet lumen defined by the elongated structure, and wherein either a diameter of the outlet lumen is greater than a diameter of the inlet lumen or the diameter of the inlet lumen is greater than the diameter of the outlet lumen.

Clause 61: The method of clause 59 or clause 60, wherein the outlet lumen defines a first flow coefficient for a mass flow rate of the fluid and the inlet lumen defines a second flow coefficient for the mass flow rate of the fluid, wherein either the first flow coefficient is less than the second flow coefficient or the second flow coefficient is less than the first flow coefficient.

Clause 62: The method of any one of clauses 59-61, wherein positioning the valve modifies a flow area within the flow path.

Clause 63: The method of clause 62, further comprising sensing, using the pressure sensor, the pressure of the fluid upstream of the flow area.

Clause 64: The method of any one of clauses 59-63, wherein at least one of the balloon or an energy emitting element is positioned on a distal portion of the elongated body, wherein the distal portion is configured to be positioned within the anatomical lumen.

Clause 65: The method of clause 64, further comprising emitting ultrasound energy using the energy emitting element.

Clause 66: The method of clause 64 or clause 65, wherein the energy emitting element is positioned within the interior volume of the balloon.

Clause 67: The method of any one of clauses 59-66, wherein the input indicative of the pressure setpoint is indicative of a dimension of the balloon.

Clause 68: The method of any one of clauses 59-67, further comprising receiving, by the control circuitry, the pressure setpoint from a user.

Clause 69: The method of any one of clauses 59-68, further comprising: determining, using the control circuitry, a pressure trend based on a plurality of sensor inputs received over a time period; and positioning the valve based on the pressure trend.

Clause 70: A method, comprising: receiving, by control circuitry, an input indicative of a pressure within an interior volume of a balloon of a catheter, wherein an elongated body of the catheter defines a flow path for a fluid flowing through the interior volume of the balloon, and wherein at least a distal portion of the catheter is configured to be positioned with an anatomical lumen of a patient; determining, by the control circuitry, a power level of an energy emitting element of the catheter based on the indicative input; and causing, using the control circuitry, the energy emitting element to emit energy based on the power level.

Clause 71: The method of clause 70, wherein the input is indicative of a pressure setpoint received from an input device.

Clause 72: The method of clause 70, further comprising: receiving, by control circuitry, a sensor input from a pressure sensor configured to sense a pressure of a fluid within the flow path, wherein the pressure of the fluid within the flow path is indicative of the pressure within the interior volume of the balloon; and determining the power level based on the sensor input.

Clause 73: The method of clause 72, further comprising: determining, by the control circuitry, a pressure trend based on a plurality of sensor inputs received over a time period; and controlling, by the control circuitry, a flow rate of a fluid flowing through the interior volume based on the pressure trend.

Clause 74: The method of any one of clauses 70-73, wherein the energy comprises ultrasound energy.

Clause 75: The method of any one of clauses 70-74, further comprising: receiving, by the control circuitry, a user input; and altering, using the control circuitry, the energy emitted by the energy emitting element based on the user input.

Clause 76: The method of any one of clauses 70-75, wherein the input indicative of the pressure within the interior volume of the balloon is indicative of a dimension of the balloon.

Clause 77: The method of any one of clauses 70-76, further comprising controlling, by the control circuitry, at least one of a valve or a pump based on the input indicative of the pressure within the interior volume of the balloon, wherein the valve or the pump is configured to control a flow rate of a fluid flowing through the interior volume.

Various examples of the disclosure have been described. Any combination of the described systems, operations, or functions is contemplated. These and other examples are within the scope of the following claims.

Further disclosed herein is the subject-matter of the following clauses:
1. A catheter system comprising:
   an elongate body configured to be positioned within a blood vessel of a patient;
   a balloon defining an interior volume;
   a sensor configured to generate a pressure signal indicative of a pressure of a fluid within a flow path, the flow path including the interior volume and at least one lumen defined by the elongate body;
   a valve fluidically coupled to the flow path and configured to define a flow area for the fluid; and
   control circuitry configured to:
      determine a pressure setpoint,
      receive the pressure signal from the sensor, and
      position the valve to adjust the flow area based on the pressure setpoint and the pressure indicated by the sensor signal.
2. The catheter system of clause 1, wherein the at least one lumen comprises an inlet lumen and an outlet lumen; wherein the flow path is defined through the inlet lumen, the interior volume, and the outlet lumen; and wherein a diameter of the outlet lumen is greater than a diameter of the inlet lumen.
3. The catheter system of clause 2, wherein the pressure signal is indicative of the pressure of the fluid within the outlet lumen.
4. The catheter system of any one of clauses 1-3, wherein the catheter system is configured to receive an input indicative of the pressure setpoint and determine the pressure setpoint using the input.
5. The catheter system of any one of clauses 1-4, wherein the valve includes:
   a restricting element configured to define the flow area; and
   a valve actuator configured to position the restricting element, wherein the control circuitry is configured to position the valve to adjust the flow area by at least causing the valve actuator to position the restricting element.
6. The catheter system of clause 5, wherein the pressure signal is indicative of pressure of the fluid upstream of the restricting element.
7. The catheter system of any one of clauses 1 to 6, further comprising a pump configured to cause the fluid to flow through the flow path, wherein the control circuitry is configured to adjust, using the pump, a flow rate of the fluid through the flow path based on the pressure setpoint and the pressure indicated by the sensor signal, and wherein the pump defines a pump outlet configured to discharge the fluid into the inlet lumen defined by the elongate body to cause the fluid to flow through the flow path.
8. The catheter system of clause 7, wherein the pump includes a centrifugal pump or a positive displacement pump.
9. The catheter system of any one of clauses 1 to 8, wherein the elongate body defines a longitudinal axis, the catheter system further comprising a therapeutic element mechanically supported by the catheter system, wherein the therapeutic element is configured to emit energy in a direction away from the longitudinal axis.
10. The catheter system of clause 9, wherein:
   the therapeutic element is configured to emit ultrasound energy; or
   a distal portion of the elongate body supports the therapeutic element; or
   the therapeutic element is positioned within the interior volume of the balloon.
11. The catheter system of clause 9 or 10, wherein the control circuitry is configured to:
   determine a power level, and cause the therapeutic element to emit the energy based on the determined power level.
12. The catheter system of clause 11, wherein the control circuitry is configured to determine the power level based on at least one of the pressure setpoint or the pressure signal indicative of the pressure of the fluid within the flow path.
13. The catheter system of any one of clauses 1 to 12, wherein the control circuitry is configured to determine the pressure setpoint based on input from a user, wherein the input is indicative of a cross-sectional dimension of the balloon perpendicular to the longitudinal axis of the elongate body.
14. A catheter system comprising:
   an elongate body;
   a balloon defining an interior volume;
   a therapeutic element mechanically supported by the elongate body, wherein the therapeutic element is configured to emit energy; and
   control circuitry configured to:
      receive an input indicative of a pressure within the interior volume of the balloon,
      determine a power level based on the indicated pressure, and
      cause the therapeutic element to emit the energy based on the determined power level.
15. The catheter system of clause 14, wherein the input includes a pressure setpoint, the catheter system further comprising a user interface configured to receive the pressure setpoint from a user, wherein the control circuitry is configured to determine the power level based on the pressure set point.
16. The catheter system of clause 14, further comprising a sensor configured to generate a pressure signal indicative of a pressure of a fluid within a flow path defined at least in part by the elongate body, wherein the pressure of the fluid within the flow path is indicative of the pressure within the interior volume of the balloon, and wherein the control circuitry is configured to:
   receive the pressure signal from the sensor, and
   determine the power level based on the pressure signal.
17. The catheter system of any one of clauses 14-16, wherein the therapeutic element is configured to emit ultrasound energy, further comprising a generator configured to provide energy to the therapeutic element.
18. The catheter system of any one of clauses 14-17, wherein the therapeutic element comprises energy control circuitry configured to cause the therapeutic element to emit the energy, and wherein the control circuitry is configured to control the energy control circuitry to cause the therapeutic element to emit the energy at the power level.
19. The catheter system of any one of clauses 14-18, wherein a dimension of the balloon varies based on the pressure within the interior volume of the balloon, and wherein the input indicative of the pressure within the interior volume of the balloon is indicative of the dimension of the balloon.
20. The catheter system of clause 19, wherein the elongate body defines a longitudinal axis, and wherein the dimension of the balloon is a cross-sectional dimension of the balloon perpendicular to the longitudinal axis.
21. The catheter system of any one of clauses 14-20, wherein the therapeutic element is positioned within the interior volume of the balloon.
22. A method, comprising:
   receiving, by control circuitry, a sensor input from a pressure sensor indicative of a pressure of a fluid within a flow path for a fluid flowing through an interior volume of a balloon, the flow path being defined by an elongated body of a catheter, wherein the elongated structure is configured to be positioned with an anatomical lumen of a patient; and
   positioning, using the control circuitry, a valve configured to throttle a flow of the fluid through the flow path based on a difference between a pressure setpoint and the pressure indicated by the sensor input.
23. A method, comprising:
   receiving, by control circuitry, an input indicative of a pressure within an interior volume of a balloon of a catheter, wherein an elongated body of the catheter defines a flow path for a fluid flowing through the interior volume of the balloon, and wherein at least a distal portion of the catheter is configured to be positioned with an anatomical lumen of a patient;
   determining, by the control circuitry, a power level of an energy emitting element of the catheter based on the indicative input; and
   causing, using the control circuitry, the energy emitting element to emit energy based on the power level.

1: A catheter system comprising:
an elongate body configured to be positioned within a blood vessel of a patient;
a balloon defining an interior volume;
a sensor configured to generate a pressure signal indicative of a pressure of a fluid within a flow path, the flow path including the interior volume and at least one lumen defined by the elongate body;
a valve fluidically coupled to the flow path and configured to define a flow area for the fluid; and
control circuitry configured to:
determine a pressure setpoint,
receive the pressure signal from the sensor, and
position the valve to adjust the flow area based on the pressure setpoint and the pressure indicated by the sensor signal.

2: The catheter system of claim 1, wherein the at least one lumen comprises an inlet lumen and an outlet lumen; wherein the flow path is defined through the inlet lumen, the interior volume, and the outlet lumen; and wherein a diameter of the outlet lumen is greater than a diameter of the inlet lumen.

3: The catheter system of claim 2, wherein the pressure signal is indicative of the pressure of the fluid within the outlet lumen.

4: The catheter system of claim 1, wherein the catheter system is configured to receive an input indicative of the pressure setpoint and determine the pressure setpoint using the input.

5: The catheter system of claim 1, wherein the valve includes:
a restricting element configured to define the flow area; and
a valve actuator configured to position the restricting element,
wherein the control circuitry is configured to position the valve to adjust the flow area by at least causing the valve actuator to position the restricting element.

6: The catheter system of claim 5, wherein the pressure signal is indicative of pressure of the fluid upstream of the restricting element.

7: The catheter system of claim 1, further comprising a pump configured to cause the fluid to flow through the flow path, wherein the control circuitry is configured to adjust, using the pump, a flow rate of the fluid through the flow path based on the pressure setpoint and the pressure indicated by the sensor signal, and wherein the pump defines a pump outlet configured to discharge the fluid into the inlet lumen defined by the elongate body to cause the fluid to flow through the flow path.

8: The catheter system of claim 7, wherein the pump includes a centrifugal pump or a positive displacement pump.

9: The catheter system of claim 1, wherein the elongate body defines a longitudinal axis, the catheter system further comprising a therapeutic element mechanically supported by the catheter system, wherein the therapeutic element is configured to emit energy in a direction away from the longitudinal axis.

10: The catheter system of claim 9, wherein:
the therapeutic element is configured to emit ultrasound energy; or
a distal portion of the elongate body supports the therapeutic element; or
the therapeutic element is positioned within the interior volume of the balloon.

11: The catheter system of claim 9, wherein the control circuitry is configured to:
determine a power level, and
cause the therapeutic element to emit the energy based on the determined power level.

12: The catheter system of claim 11, wherein the control circuitry is configured to determine the power level based on at least one of the pressure setpoint or the pressure signal indicative of the pressure of the fluid within the flow path.

13: The catheter system of claim 1, wherein the control circuitry is configured to determine the pressure setpoint based on input from a user, wherein the input is indicative of a cross-sectional dimension of the balloon perpendicular to the longitudinal axis of the elongate body.

14: A catheter system comprising:
an elongate body;
a balloon defining an interior volume;
a therapeutic element mechanically supported by the elongate body, wherein the therapeutic element is configured to emit energy; and
control circuitry configured to:
receive an input indicative of a pressure within the interior volume of the balloon,
determine a power level based on the indicated pressure, and
cause the therapeutic element to emit the energy based on the determined power level.

15: The catheter system of claim 14, wherein the input includes a pressure setpoint, the catheter system further comprising a user interface configured to receive the pressure setpoint from a user, wherein the control circuitry is configured to determine the power level based on the pressure set point.

16: The catheter system of claim 14, further comprising a sensor configured to generate a pressure signal indicative of a pressure of a fluid within a flow path defined at least in part by the elongate body, wherein the pressure of the fluid within the flow path is indicative of the pressure within the interior volume of the balloon, and wherein the control circuitry is configured to:
receive the pressure signal from the sensor, and
determine the power level based on the pressure signal.

17: The catheter system of claim 14, wherein the therapeutic element is configured to emit ultrasound energy, further comprising a generator configured to provide energy to the therapeutic element.

18: The catheter system of claim 14, wherein the therapeutic element comprises energy control circuitry configured to cause the therapeutic element to emit the energy, and wherein the control circuitry is configured to control the energy control circuitry to cause the therapeutic element to emit the energy at the power level.

19: The catheter system of claim 14, wherein a dimension of the balloon varies based on the pressure within the interior volume of the balloon, and wherein the input indicative of the pressure within the interior volume of the balloon is indicative of the dimension of the balloon.

20: The catheter system of claim 19, wherein the elongate body defines a longitudinal axis, and wherein the dimension of the balloon is a cross-sectional dimension of the balloon perpendicular to the longitudinal axis.

21: The catheter system of claim 14, wherein the therapeutic element is positioned within the interior volume of the balloon.

22: A method, comprising:
receiving, by control circuitry, a sensor input from a pressure sensor indicative of a pressure of a fluid within a flow path for a fluid flowing through an interior volume of a balloon, the flow path being defined by an elongated body of a catheter, wherein the elongated structure is configured to be positioned with an anatomical lumen of a patient; and
positioning, using the control circuitry, a valve configured to throttle a flow of the fluid through the flow path based on a difference between a pressure setpoint and the pressure indicated by the sensor input.

23: A method, comprising:
receiving, by control circuitry, an input indicative of a pressure within an interior volume of a balloon of a catheter, wherein an elongated body of the catheter defines a flow path for a fluid flowing through the interior volume of the balloon, and wherein at least a distal portion of the catheter is configured to be positioned with an anatomical lumen of a patient;
determining, by the control circuitry, a power level of an energy emitting element of the catheter based on the indicative input; and
causing, using the control circuitry, the energy emitting element to emit energy based on the power level.
